(19) United States
(12) Patent Application Publication
(10) Pub. No.: US 2026/0315599 A1
(43) Pub. Date: Oct. 15, 2026

(54) SMALL MOLECULE MODIFIED BORON CONTAINING COMPOUNDS AND METHODS OF USE THEREOF

(71) Applicant: Scott L. Cohen, Lynnfield, MA (US)

(72) Inventor: Scott L. Cohen, Lynnfield, MA (US)

(21) Appl. No.: 19/090,375

(22) Filed: Mar. 25, 2025

Related U.S. Application Data
(60) Provisional application No. 63/569,726, filed on Mar. 25, 2024.

(51) Int. Cl. A61K 41/00; A61K 47/54; A61P 35/00

(52) U.S. Cl. CPC A61K 41/0095; A61K 47/545; A61P 35/00

(57) ABSTRACT

The present invention is directed to novel compounds and related bi-modal therapeutic methods of treating cancer(s), immunological disorders, and other diseases. The present invention has identified novel pathway targets that are intracellular and have remained unexplored as targets for boron neutron capture therapy. By combining highly effective small molecule targeting of intracellular pathway proteins with bi-modal boron neutron capture therapy functionality, the present invention provides powerful dual acting therapeutic compounds for the treatment of cancer(s), immunological disorders, and other diseases, also referred to herein as pathway interacting borylated small molecules or PIBS. Moreover, these novel compounds comprise borylated small molecules that bi-modally treat cancer(s), immunological disorders, and other diseases by targeting intracellular disease pathways for modification, and which also serve to capture neutrons for boron neutron capture therapy.

### RELATED APPLICATIONS

This application is claims priority to U.S. Provisional Patent Application No. 63/569,726, filed on Mar. 25, 2024, the entirety of which is incorporated herein by reference.

### BACKGROUND OF THE INVENTION

Cancer is the second leading cause of death next to coronary disease worldwide. Millions of people die from cancer every year and in the United States alone cancer kills well over a half-million people annually, with 1,688,780 new cancer cases diagnosed in 2017 (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. In particular, carcinomas of the lung (18.4% of all cancer deaths), breast (6.6% of all cancer deaths), colorectal (9.2% of all cancer deaths), liver (8.2% of all cancer deaths), and stomach (8.2% of all cancer deaths) represent major causes of cancer death for both sexes in all ages worldwide (GLOBOCAN 2018). These and virtually all other carcinomas share a common lethal feature in that they metastasize to sites distant from the primary tumor and with very few exceptions. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Sadly, many cancer patients experience a recurrence of their disease.

Although cancer therapy has improved over the past decades and survival rates have increased, the heterogeneity of cancer still demands new therapeutic strategies. This is especially true in treating solid tumors at anatomically crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients' quality of life. Over the years, such detrimental effects have prompted the development of newer techniques like Neutron Capture Therapy (NCT).

NCT is a promising form of radiation therapy. It is a technique that selectively kills tumor cells using boron compound while sparing the normal cells.

Boron Neutron Capture Therapy (BNCT) relies on the propensity of non-radioactive ¹⁰B isotope to absorb epithermal neutrons that fall into the low energy range of 0.5 keV<En<30 keV. Following neutron capture, boron atom undergoes a nuclear fission reaction giving rise to an alpha-particle and a recoiled lithium nucleus (7Li) as follows: ¹⁰B+n→⁷Li+⁴He

The alpha particle deposits high energy i.e. 150 keV/μm along their short path essentially restricted to a single cell diameter that results in a double strand DNA breaks followed by cancer cell death by apoptosis. Thus BNCT integrates a concept of both chemotherapy, targeted therapy and the gross anatomical localization of traditional radiotherapy.

What is more is that even though the conceptual techniques of NCT, and specifically Boron Neutron Capture Therapy (BNCT), are well known, the technological limitations associated with this type of treatment have slowed progress. During the early investigations using the research reactors of MIT in 1960's, several dozens of patients were treated using disodium decahydrodecaborate, which was considered less toxic than simple boron compounds used previously yet capable of delivering more boron to the cell. Unfortunately, BNCT studies were halted in the USA due to the severe brain necrosis in the patients undergoing BNCT and the potential harm of using nuclear reactors.

However, although there has been a slow resurgence of BNCT that has taken place over the last 50 years, it will be readily apparent to those of ordinary skill in the art that novel treatment paradigms of BNCT are needed for the treatment of cancers and immunological diseases.

### SUMMARY OF THE INVENTION

Accordingly, the present invention is directed to novel compounds and related bi-modal therapeutic methods of treating cancer(s), immunological disorders, and other diseases. The present invention has identified novel pathway targets that are intracellular and have remained unexplored as targets for boron neutron capture therapy. By combining highly effective small molecule targeting of intracellular pathway proteins with bi-modal boron neutron capture therapy functionality, the present invention provides powerful dual acting therapeutic compounds for the treatment of cancer(s), immunological disorders, and other diseases. The present invention is referred to herein as “Pathway Interacting Borylated Small molecules” or PIBS. Moreover, these novel compounds comprise borylated small molecules that bi-modally treat cancer(s), immunological disorders, and other diseases by targeting intracellular disease pathways for modification, and which also serve to capture neutrons for boron neutron capture therapy.

As such, one aspect of the present invention provides a compound of formula A:

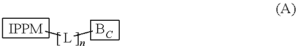

wherein
n is 0 or 1;
IPPM is a molecular structure identified to bind to and modify an intracellular protein pathway at the protein or the DNA encoding the protein associated with the treatment of cancer(s), immunological disorders, and other diseases;
B_{C} is a boron component molecule suitable for use in Boron Neutron Capture Therapy (BNCT); and
L is a linking structure that links IPPM to B_{C}, wherein the identification and selection of the linking position on the IPPM is made to ensure retention of substantial activity of the IPPM to bind and modify the intracellular protein pathway.

Another aspect of the present invention provides a pharmaceutical composition comprising a compound of the present invention, or a pharmaceutically acceptable derivative thereof, and a pharmaceutically acceptable carrier, adjuvant, or vehicle.

Another aspect of the present invention provides a method of treating an intracellular protein mediated disorder, disease, or condition in a patient comprising administering to said patient a compound of the invention.

### DETAILED DESCRIPTION OF THE INVENTION

Given the current deficiencies associated with NCT, identifying novel pathway targets that offer bi-modal therapeutic advantages over existing therapies offers a major shift in the treatment paradigm for cancer(s), immunological disorders, and other diseases. Utilizing a plurality of treatment modalities in these treatments provides novel disease treatment that are more effective with reduced side effects.

As such, the present invention is directed to novel compounds and related bi-modal therapeutic methods of treating cancer(s), immunological disorders, and other diseases. The present invention has identified novel pathway targets that are intracellular and have remained unexplored as targets for boron neutron capture therapy. By combining highly effective small molecule targeting of intracellular pathway proteins with bi-modal boron neutron capture therapy functionality, the present invention provides powerful dual acting therapeutic compounds for the treatment of cancer(s), immunological disorders, and other diseases, also referred to herein as pathway interacting borylated small molecules or PIBS. Moreover, these novel compounds comprise borylated small molecules that bi-modally treat cancer(s), immunological disorders, and other diseases by targeting intracellular disease pathways for modification, and which also serve to capture neutrons for boron neutron capture therapy.

In particular embodiments, these treatments offer borylated small molecules that both modify a cancer pathway to kill cancer (e.g., upregulate P53 activity, inhibit PI3Ka, inhibit KRAS) as well as destroying cancer cell/tissue with an alpha particle delivered by boron targeting an intracellular protein or cleaving cancer DNA. The PIBS of the present invention have high targeting specificity and cytotoxicity.

The present invention, including compounds and methods will be described with reference to the following definitions that, for convenience, are set forth below. Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise specified, the below terms used herein are defined as follows:

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “about” is used herein in reference to the degree or extent of the term which it modifies, and that such extent may be 100% or near to but not exactly 100% of the modified term; industry accepted standards will assist in defining the quantitative aspects of how “near” 100% is defined. In particular embodiments, the term “about” indicates ±3%, ±2%, ±1% or ±0.5%.

As used herein, the term “active metabolite or residue thereof” means that a metabolite or residue thereof is also a binder of a protein, a mutant thereof, or the DNA encoding these proteins.

The term “aliphatic” or “aliphatic group”, as used herein, describes a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) describes a monocyclic C3-C6 hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkylene” describes a bivalent alkyl group. An “alkylene chain” is a polymethylene group, i.e., —(CH₂)n-, wherein n is a positive integer, for example, from 1 to 6, from 1 to 4, from 1 to 3, from 1 to 2, or from 2 to 3. A substituted alkylene chain is a polymethylene group in which one or more methylene hydrogen atoms are replaced with a substituent.

The term “alkenylene” describes a bivalent alkenyl group. A substituted alkenylene chain is a polymethylene group containing at least one double bond in which one or more hydrogen atoms are replaced with a substituent.

The term “aryl” used alone or as part of a larger moiety as in “aralkyl,” “aralkoxy,” or “aryloxyalkyl,” describes monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” describes an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

As used herein, the term “binder” or “ligand” is defined as a compound that binds to a protein or DNA encoding the protein with measurable affinity. In certain embodiments, a compound of the present invention has a binding constant of less than about 50 M, less than about 1 μM, less than about 500 nM, less than about 100 nM, less than about 10 nM, or less than about 1 nM.

As used herein, the term “bivalent C1-8 (or C1-6) saturated or unsaturated, straight or branched, hydrocarbon chain”, describes bivalent alkylene, alkenylene, and alkynylene chains that are straight or branched as defined herein.

The term “borylation” describes reactions that produce an organoboron compound through functionalization of aliphatic and aromatic C—H bonds.

The term “borylated” describes a compound which has undergone a borylation reaction.

As used herein, the term “bridged bicyclic” describes any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents. Additionally or alternatively, any substitutable nitrogen of a bridged bicyclic group is optionally substituted. Exemplary bridged bicyclics include:

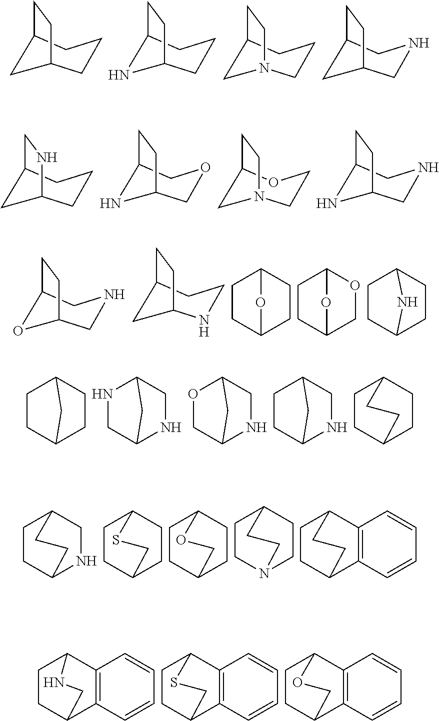

The term “compound” describes and encompasses the chemical compound, as well as, whether explicitly stated or not, (and unless the context makes clear that the following are to be excluded): amorphous and crystalline forms of the compound, including polymorphic forms, where these forms may be part of a mixture or in isolation; free acid and free base forms of the compound, which are typically the forms shown in the structures provided herein; isomers of the compound, which describes optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, for example, pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above.

As used herein, the term “cyclopropylenyl” describes a bivalent cyclopropyl group of the following structure:

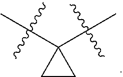 [SMILES: *C1(*)CC1]

As used herein, the term “covalent cysteine modifier moiety” or “covalent cysteine modifier substituent” is an electrophilic substituent is capable of forming a covalent bond with a cysteine residue, which may be added to any one of the compounds of the invention. The covalent bond formed between the electrophilic substituent and the sulfhydryl group of the cysteine may be a reversible or irreversible bond. In certain examples of this warhead moiety, the substituent is:

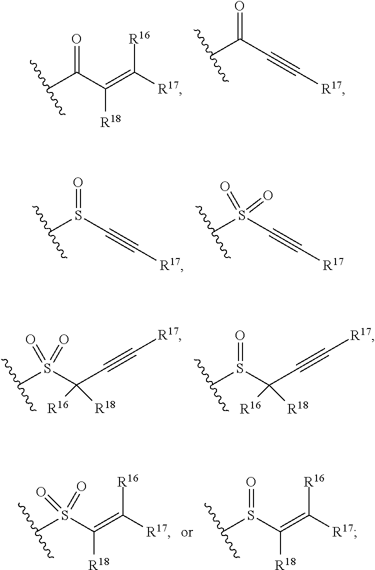

and
wherein R¹⁶, R¹⁷, and R¹⁸ are independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

The term “halogen” means F, Cl, Br, or I.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 pi electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” describes an alkyl group substituted by a heteroaryl, wherein the alkyl and heteroaryl portions independently are optionally substituted.

The term “heteroatom” describes one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

As used herein, the terms “heterocycle,” “heterocyclyl,” “heterocyclic radical,” and “heterocyclic ring” are used interchangeably and refer to a stable 5- to 9-membered monocyclic or 7- to 11-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” describes an alkyl group substituted by a heterocyclyl, wherein the alkyl and heterocyclyl portions independently are optionally substituted.

The term “including” as well as other forms, such as “include,” “includes,” and “included,” is not limiting.

The terms “inhibit” or “inhibiting” of as used herein means to reduce by a measurable amount, or to prevent entirely.

The term “lower alkyl” describes a C1-4 straight or branched alkyl group.

Exemplary lower alkyl groups are methyl, ethyl, propyl, isopropyl, butyl, isobutyl, and tert-butyl.

The term “lower haloalkyl” describes a C1-4 straight or branched alkyl group that is substituted with one or more halogen atoms.

The term “mammal” describes any organism classified as a mammal, including mice, rats, rabbits, dogs, cats, cows, horses and humans. In one embodiment of the invention, the mammal is a mouse. In another embodiment of the invention, the mammal is a human.

The term “mass-tag” as used herein describes any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably modulate,” as used herein, describes a measurable change in a protein activity between a sample comprising a compound of the present invention, or composition thereof, and a protein, and an equivalent sample comprising a protein, in the absence of said compound, or composition thereof.

The terms “metastatic cancer” and “metastatic disease” mean cancers that have spread to regional lymph nodes or to distant sites, and are meant to include stage D disease under the AUA system and stage T×N×M+ under the TNM system.

The terms “molecular recognition” and “binds” mean a chemical event in which a host molecule is able to form a complex with a second molecule (i.e. the guest). This process occurs through covalent or non-covalent chemical bonds. Non-covalent chemical bonds include but are not limited to, hydrogen bonding, hydrophobic interactions, and ionic interaction.

The term “neutron capture agent” describes a stable non-reactive chemical isotope which, when activated by neutrons produces alpha particles.

The term “neutron capture therapy” describes a noninvasive therapeutic modality for treating locally invasive malignant tumors and other immunological disorders and disease by irradiating a neutron capture agent with neutrons. Neutron Capture Therapy (NCT) is a promising form of radiation therapy. It is a technique that selectively kills tumor cells, e.g., using boron compounds, while sparing the normal cells. For example, BNCT relies on the propensity of non-radioactive ¹⁰B isotope to absorb epithermal neutrons that fall into the low energy range of 0.5 keV<En<30 keV. Following neutron capture, boron atom undergoes a nuclear fission reaction giving rise to an alpha-particle and a recoiled lithium nucleus (7Li) as follows:

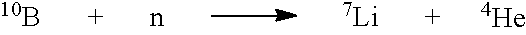

The alpha particle deposits high energy i.e. 150 keV/μm along their short path essentially restricted to a single cell diameter that results in a double strand DNA breaks followed by cancer cell death by apoptosis.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, describes compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N3; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N3, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}. —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*2))₂-3O—, or —S(C(R*2))₂-3S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂-3O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C1-4 aliphatic, —CH₂Ph, —O(CH₂)₀-1Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{t} is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH2, —NHR^{•}, —NR^{•}2, or —NO₂, wherein each R^{•} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, the term “partially unsaturated” describes a ring moiety that includes at least one double or triple bond. The term “partially unsaturated” is intended to encompass rings having multiple sites of unsaturation, but is not intended to include aryl or heteroaryl moieties, as herein defined.

The language “pharmaceutically acceptable” describes a non-toxic, inert, and/or composition that is physiologically compatible with humans or other mammals.

As used herein, the term “pharmaceutically acceptable salt” describes those salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharm. Sci. 1977 66:1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C1-4alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “provided compound” describes any genus, subgenus, and/or species set forth herein.

The term “secondary label” as used herein describes moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The term “patient,” as used herein, describes an animal, preferably a mammal, and most preferably a human, to which a therapeutic is administered.

The term “pharmaceutically acceptable carrier, adjuvant, or vehicle” describes a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” describes any non-toxic salt, ester, salt of an ester or other derivative of a compound of this invention that, upon administration to a recipient, is capable of providing, either directly or indirectly, a compound of this invention or an active metabolite or residue thereof.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “unsaturated,” as used herein, describes that a moiety has one or more units of unsaturation.

### II. Compounds and Compositions of the Present Invention

The compounds and compositions of the present invention target intracellular pathway proteins for modification with bi-modal boron neutron capture therapy functionality. These pathway interacting borylated small molecules (“PIBS”) provide powerful dual acting therapeutic compounds for the treatment of cancer(s), immunological disorders, and other diseases, i.e., bi-modally treat cancer(s), immunological disorders, and other diseases by targeting intracellular disease pathways for modification, and which also serve to capture neutrons for boron neutron capture therapy.

### A. Compounds

The pathway interacting borylated small molecules (“PIBS”) of the present invention comprise a compound structure of a Boron Component (B_{C}) linked to an Intracellular Protein Pathway Modifier (IPPM) by a direct bond or a Linker (L):

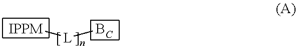

wherein
n is 0 or 1;
IPPM is a molecular structure identified to bind to and modify an intracellular protein pathway at the protein or the DNA encoding the protein (e.g., associated with the treatment of cancer(s), immunological disorders, and other diseases);
B_{C} is a boron component molecule suitable for use in Boron Neutron Capture Therapy (BNCT); and
L is a linking structure that links IPPM to B_{C}, wherein the identification and selection of the linking position on the IPPM is made to ensure retention of substantial activity of the IPPM to bind and modify the intracellular protein pathway.

In certain embodiments of the present invention, L is a covalent bond or —S—, —O—, —C(O)—, —C(S)—, —CZ¹₂—, —CZ¹F—, —CF₂—, —NZ¹—, or —S(O)₂—, or a bivalent, saturated or unsaturated, straight or branched C₁₋₂₀ hydrocarbon chain, wherein 0-6 methylene units of L are independently and optionally replaced with —S—, —O—, —C(O)—, —C(S)—, —CZ¹₂—, —CZ¹F—, —CF₂—, —NZ¹—, or —S(O)₂—, or by —C(D)(H)—, —C(D)₂-, —CZ¹F—, —CF₂—, -Cy-, —O—, —N(Z¹)—, —Si(Z¹)₂—, —Si(OH)(Z¹)—, —Si(OH)₂—, —P(O)(OZ¹)—, —P(O)(Z¹)—, —P(O)(NZ¹₂)—, —S—, —OC(O)—, —C(O)O—, —C(O)—, —S(O)—, —S(O)₂—, —N(Z¹)S(O)₂—, —S(O)₂N(Z¹)—, —N(Z¹)C(O)—, —C(O)N(Z¹)—, —OC(O)N(Z¹)—, —N(Z¹)C(O)O—,

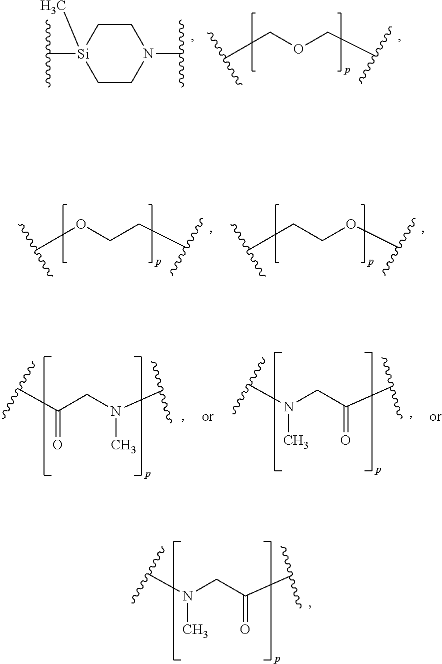

wherein:
   each p is independently 0, 1, 2, 3, 4, 5, 6, 7, 8, 9, or 10;
   each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
   each Z¹ is independently selected from the group consisting of hydrogen, deuterium, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃,
   and wherein:
      each Z¹ and R is independently selected from hydrogen, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or
      two Z¹ or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In certain embodiments of the present invention, B_{C} is selected from B₁₂H₁₁, B(OH)₂, Bpin, B(OZ)₂, wherein each Z¹ is as defined above. In certain embodiments, B_{C} is dodecaborane, which is also referred as B₁₂H₁₁:

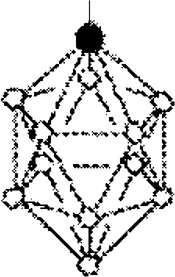

wherein the open circles represents BH and the closed circle represents B. In certain other embodiments, B_{C} is selected from a carborane with mixed amounts of boron and carbon selected from:

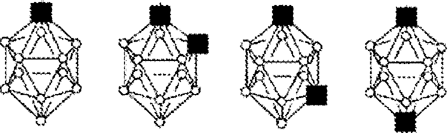

wherein the open circles represent BH and closed square represents carbon (substituted by H or L). In certain other embodiments, B_{C} is selected from a borane selected from the group consisting of hyperclosoborane (BₙHₙ), closoborane ([BₙHₙ]²⁻), nidoborane (BₙHₙ₊₄), arachnoborane (BₙHₙ₊₆), and hyphoborane (BₙHₙ₊₈).

In certain embodiments, boron may be comprised of any isotope of Boron and in particular embodiments, is enriched in ¹⁰B.

In certain embodiments -L-B_{C} is selected from the group consisting of:

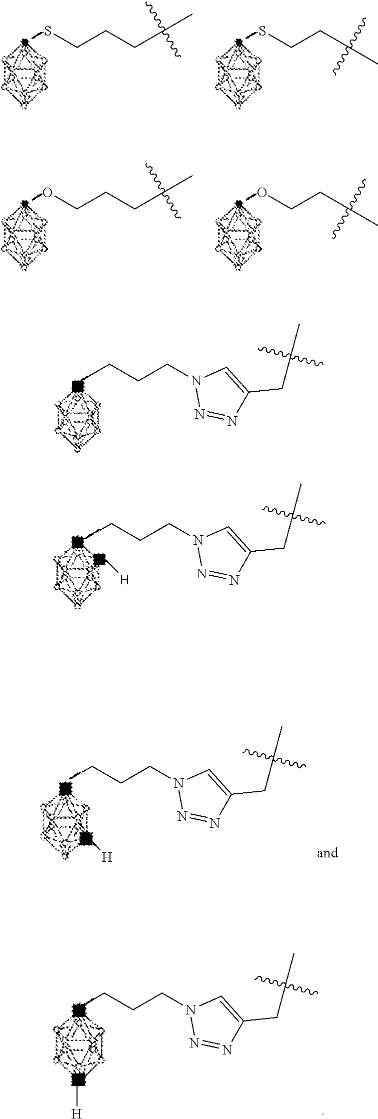

In certain embodiments of the present invention, the intracellular protein participates in a cancer regulating pathway. In certain embodiments, the cancer regulating pathway may include, but is not limited to one or more of the following known signaling pathways that are known to participate in cancer regulation:
i) RAS/MAPK pathway
ii) PI3K/AKT/mTOR pathway
iii) Notch pathway
iv) Wnt/β-catenin pathway
v) P53 pathway
vi) TGF-β pathway
vii) Hippo YAP/TAZ pathway

In particular embodiments, the IPPM binds preferentially to an intracellular protein or molecule (e.g., gene/DNA encoding the protein) but may also bind to an extracellular protein or molecule (e.g., gene/DNA encoding the protein).

### A. Exemplary Intracellular Pathway Targets

Although each of these identified intracellular pathways is suitable for the compounds of the present invention, wherein identified target molecules may be re-structured by linking the identified IPPM to a boron component either directly or through a linker. The P53 pathway, the PI3K pathway and the KRAS pathway are used herein as varied examples to show the full scope of the pathway targets of the present invention.

i. P53 Pathway

One embodiment of the present invention is a bi-modal compound of the present invention suitable to target and modify a P53 protein with the IPPM and derived from PCT Application WO2017143291, with a selectively identified linkage to a Boron component, Be of the present invention, wherein the target protein-binding moiety is derived, for example, from the compound of the formula (I):

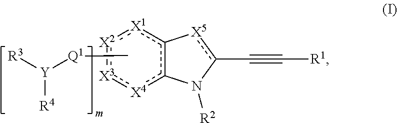

wherein:
   each bond denoted by a solid and dashed line is independently a single bond or a double bond;
   X¹ is CR⁵, CR⁵R⁶, N, NR⁵, O, S, C═O, C═S, or a carbon atom connected to Q¹;
   X² is CR⁷, CR⁷R⁸, N, NR⁷, O, S, C═O, C═S, or a carbon atom connected to Q¹;
   X³ is CR⁹, CR⁹R¹⁰, N, NR⁹, O, S, C═O, C═S, or a carbon atom connected to Q¹;
   X⁴ is CR¹¹, CR¹¹R¹², N, NR¹¹, O, S, C═O, C═S, or a carbon atom connected to Q¹;
   X⁵ is CR¹³, N, or NR¹³;
   and wherein at least one of X¹, X², X³, and X⁴ is a carbon atom connected to Q;
   Q¹ is C═O, C═S, C═CR¹⁴R¹⁵, C═NR⁴, alkylene, alkenylene, or alkynylene, each of which is independently substituted or unsubstituted, or a bond;
   m is 1, 2, 3, or 4;
   Y is N, O, or absent;
   R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen, and wherein each R¹ may be further substituted with the substituent -L-B_{c};
   each R³ and R⁴ is independently, —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent, and wherein each R³ and R⁴ taken together or independently may be further substituted with the substituent -L-B_{c};
   each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR^{2′}R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
   each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
   each R²¹ and R²² is independently alkyl, alkenyl, alkynyl, aryl, heteroaryl, or heterocyclyl, each of which is independently substituted or unsubstituted, or hydrogen;
   each R²³ and R²⁴ is independently alkyl, alkenyl, alkynyl, aryl, heteroaryl, or heterocyclyl, each of which is independently substituted or unsubstituted, or hydrogen; and
with the proviso that at least one of R¹, R³, R⁴, or R³ and R⁴ taken together are substituted with the substituent -L-B_{c},
or a pharmaceutically-acceptable salt thereof. In certain embodiments, -L-B_{c} is defined hereinabove (e.g., as described under Formula A).

In certain embodiments, one R¹ is further substituted with the substituent -L-B_{c}.

In certain embodiments, one R³, R⁴, or R³ and R⁴ taken together is substituted with the substituent -L-B_{c}.

In certain embodiments of Formula I, X³ is a carbon atom connected to Q¹, and Y is N or O.

In certain embodiments of Formula I, m is 1, and Y is N.

In certain embodiments of Formula I, the compound is of the formula:

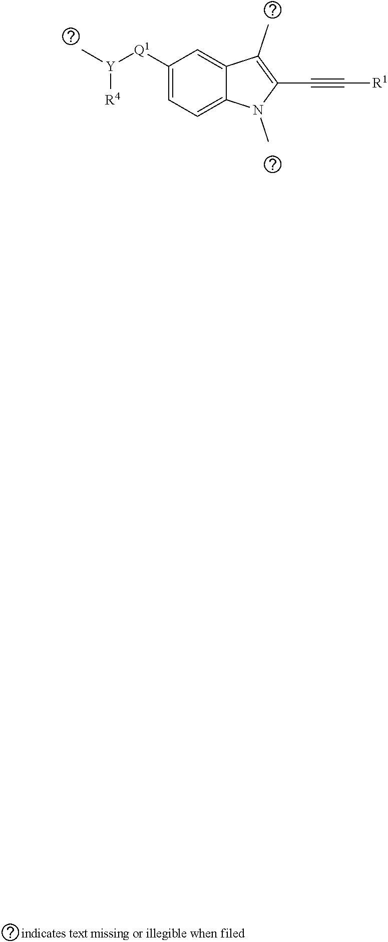

In certain embodiments of Formula I, R¹ is alkyl, alkenyl, —C(O)R¹⁶, —C(O)OR¹⁶, or —C(O)NR¹⁶R¹⁷.

In certain embodiments of Formula I, R¹ is alkyl substituted with NR¹⁶R¹⁷.

In certain embodiments of Formula I, the compound is of the formula:

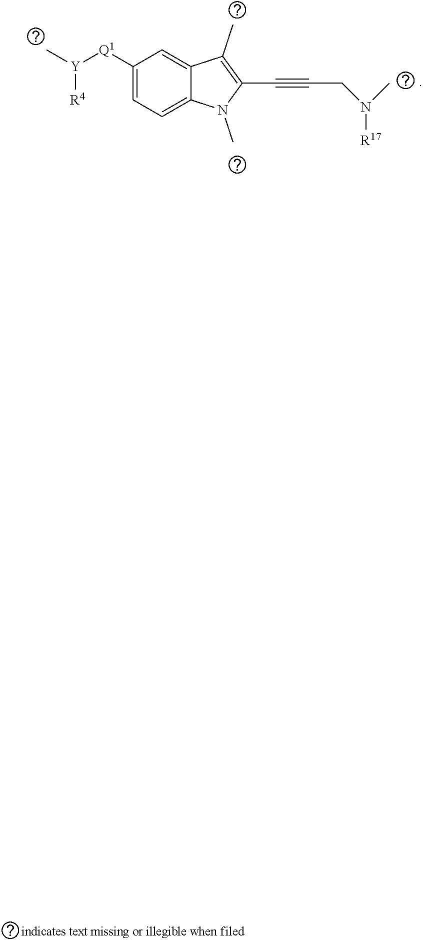

In certain embodiments of Formula I, each R and R is independently alkyl, alkenyl, aryl, heteroaryl, heterocyclyl, or hydrogen.

In certain embodiments of Formula I, R¹⁶ is hydrogen or alkyl.

In certain embodiments of Formula I, R¹⁷ is aryl, heteroaryl, or heterocyclyl, each of which is independently substituted or unsubstituted with halogen, alkyl, or hydroxyl.

In certain embodiments of Formula I, Q¹ is C═O, C═NR¹⁴, a bond, alkylene, or alkenylene.

In certain embodiments of Formula I, Q¹ is C₁-alkylene.

In certain embodiments of Formula I, R² is hydrogen or alkyl.

In certain embodiments of Formula I, R² is alkyl.

In certain embodiments of Formula I, R² is cycloalkyl.

In certain embodiments of Formula I, R¹³ is alkyl, alkenyl, hydrogen, or halogen.

In certain embodiments of Formula I, R¹³ is hydrogen.

In certain embodiments of Formula I, R¹³ is —C(O)H, —CH₂C(O)H, —CH₂C(O)OH, —C(O)OH, —CH₂NHCH₃, —CH₂NH₂, or —CH₂N(CH₃)₂.

In certain embodiments of Formula I, R³ is

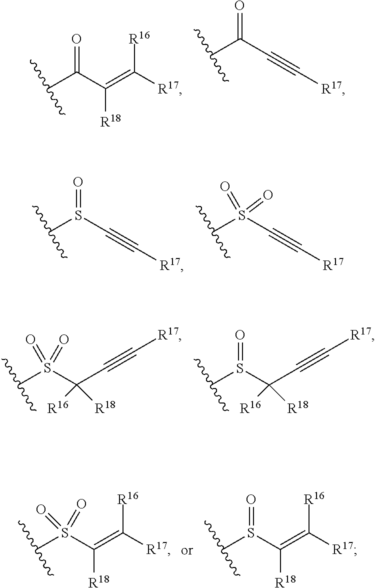

and
R¹⁶, R¹⁷, and R¹⁸ are independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In certain embodiments of Formula 1, R³ is

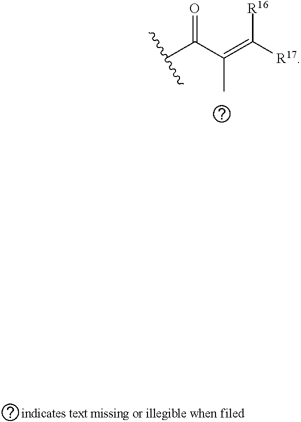

In certain embodiments, R¹⁶ is hydrogen, and R¹⁸ is hydrogen. In certain embodiments, R¹⁶ is hydrogen, R¹⁷ is hydrogen, and R¹⁸ is unsubstituted methyl. In certain embodiments, R¹⁶ is hydrogen, R¹⁷ is hydrogen, and R¹⁸ is —CN.

In certain embodiments of Formula 1, R³ and R⁴ are each independently alkyl, alkenyl, aryl, heteroaryl, heterocyclyl, or hydrogen; and wherein each R³ and R⁴ taken together or independently is further substituted with the substituent -L-B_{c}

In certain embodiments of Formula I, R⁴ is alkyl substituted with aryl; and further substituted with the substituent -L-B_{c}.

In certain embodiments of Formula 1, R³ and R⁴ together with the nitrogen atom to which R³ and R⁴ are bound form a ring, wherein the ring is further substituted with the substituent -L-B_{c}.

In certain embodiments of Formula 1, R³ and R⁴ together with the nitrogen atom to which R³ and R⁴ are bound form a heterocycle, wherein the heterocycle is substituted by at least one substituent.

In certain embodiments of Formula I, X¹ is a carbon atom connected to Q¹.

In certain embodiments of Formula 1, m is 1.

In certain embodiments of Formula 1, the compound is of the formula:

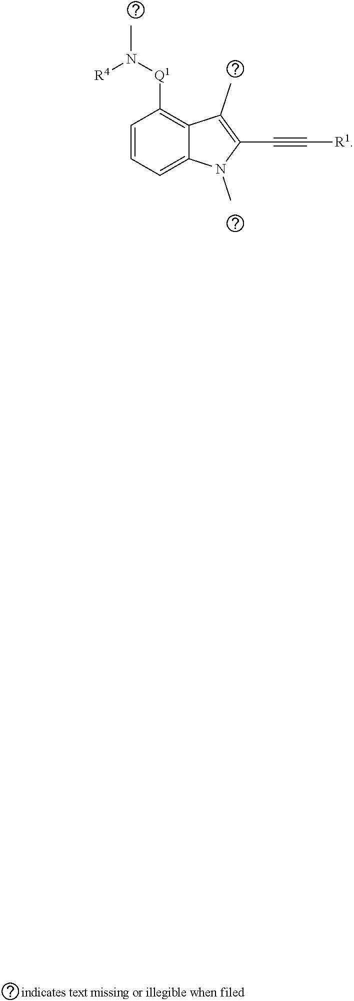

In certain embodiments of Formula I, R¹ is alkyl, alkenyl, —C(O)R¹⁶, —C(O)OR¹⁶, or —C(O)NR¹⁶R¹⁷.

In certain embodiments of Formula I, R¹ is alkyl substituted with NR¹⁶R¹⁷.

In certain embodiments of Formula I, the compound is of the formula:

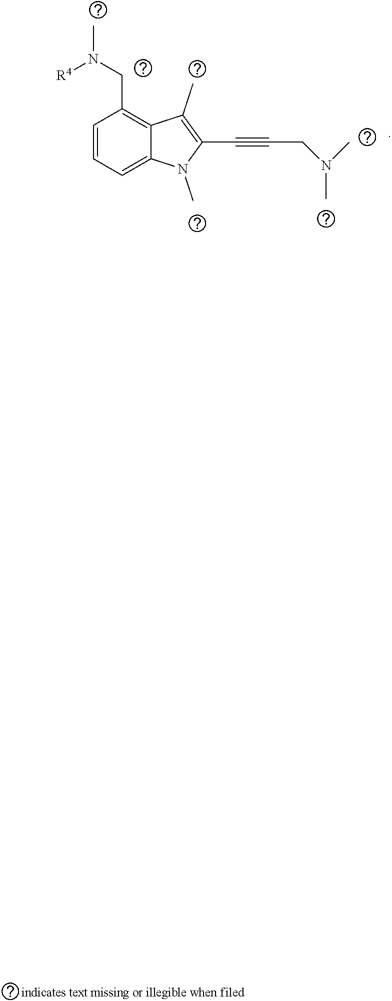

In certain embodiments of Formula I, each R¹⁶ and R¹⁷ is independently alkyl, alkenyl, aryl, heteroaryl, heterocyclyl, or hydrogen.

In certain embodiments of Formula I, R¹⁶ is hydrogen or alkyl.

In certain embodiments of Formula I, R¹⁷ is aryl, heteroaryl, or heterocyclyl, each of which is independently substituted or unsubstituted with alkyl, each of which is independently substituted or unsubstituted with halogen, alkyl, or hydroxyl.

In certain embodiments of Formula I, Q¹ is C═O, C═NR¹⁴, a bond, alkylene, or alkenylene.

In certain embodiments of Formula I, Q¹ is C1-alkylene.

In certain embodiments of Formula I, R² is hydrogen or alkyl.

In certain embodiments of Formula I, R¹³ is alkyl, alkenyl, hydrogen, or halogen.

In certain embodiments of Formula I, R¹³ is hydrogen.

In certain embodiments of Formula I, R³ and R⁴ are each independently alkyl, alkenyl, aryl, heteroaryl, heterocyclyl, or hydrogen; and wherein each R³ and R⁴ taken together or independently is further substituted with the substituent -L-B_{c}.

In certain embodiments of Formula I, R³ and R⁴ together with the nitrogen atom to which R³ and R⁴ are bound form a ring, wherein the ring is further substituted with the substituent -L-B_{c}.

In certain embodiments of Formula I, R³ and R⁴ together with the nitrogen atom to which R³ and R⁴ are bound form a heterocycle, wherein the heterocycle is substituted by at least one substituent; and wherein each R³ and R⁴ taken together or independently is further substituted with the substituent -L-B_{c}.

In certain embodiments of Formula I, R³ and R⁴ together with the nitrogen atom to which R³ and R⁴ are bound form a heterocycle, wherein the heterocycle is optionally substituted by a substituted heterocycle; and is further substituted with the substituent -L-B_{c}.

In certain embodiments, the compound of Formula I comprises the following formula:

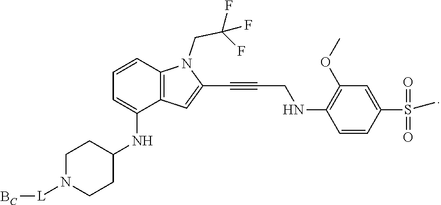

In certain embodiments, the compound of Formula I comprises the following formula:

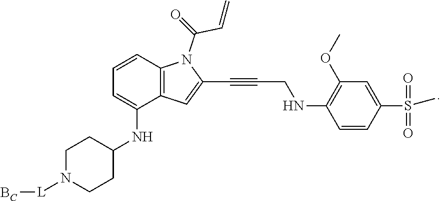

In certain embodiments, the compound of Formula I comprises the following formula:

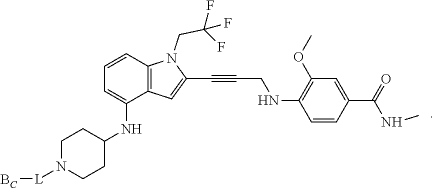

In certain embodiments, the compound of Formula I comprises the following formula:

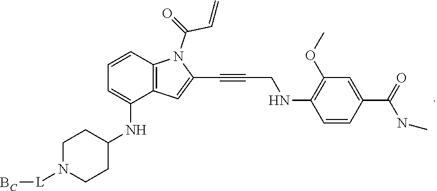

In certain embodiments, the compound of Formula I comprises the following formula:

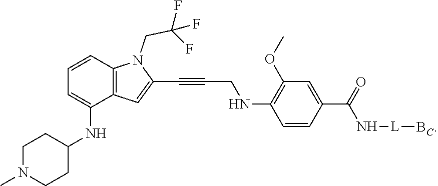

In certain embodiments, the compound of Formula I comprises the following formula:

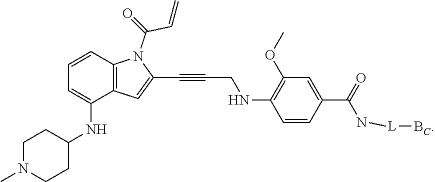

In certain embodiments of Formula I, L-B_{C} is selected from the group consisting of:

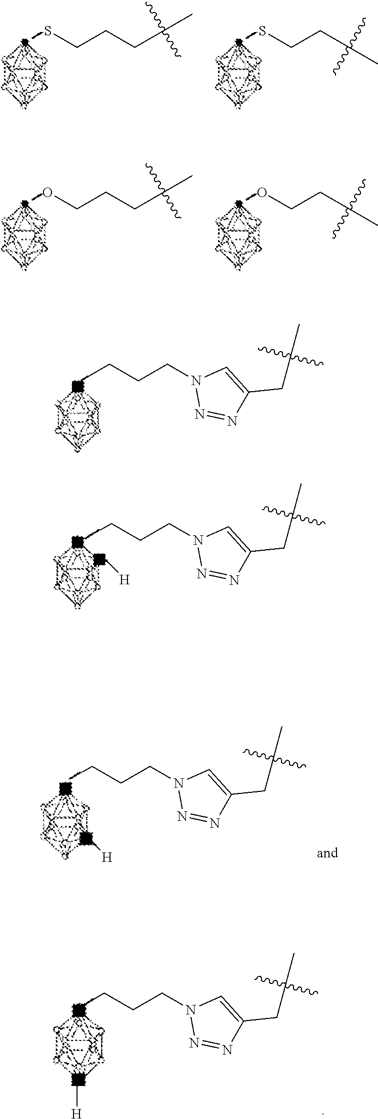

For example, in certain embodiments, the compound of Formula I comprises the following formula:

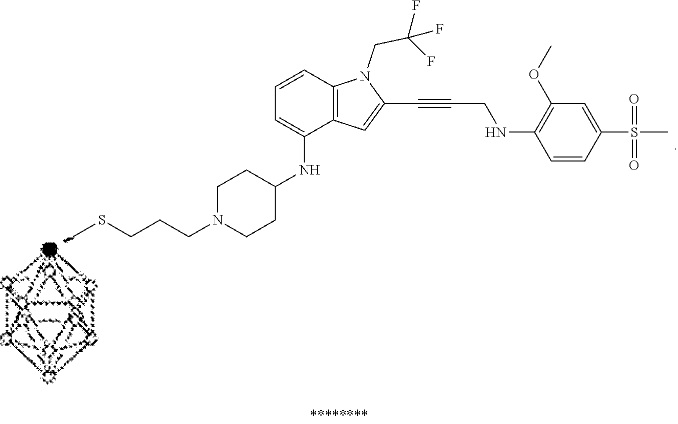

Another embodiment of the present invention is a bi-modal compound of the present invention suitable to target and modify a P53 protein with the IPPM and derived from PCT Application WO2021061643, with a selectively identified linkage to a Boron component of the present invention, wherein the target protein-binding moiety is derived, for example, from the compound of the formula (II):

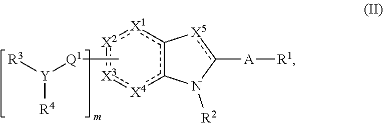

wherein:
   each is independently a single bond or a double bond;
   X¹ is CRS, CR⁵R⁶, N, NR⁵, O, S, C═O, C═S, or a carbon atom connected to Q¹;
   X² is CR⁷, CR⁷R⁸, N, NR⁷, O, S, C═O, C═S, or a carbon atom connected to Q¹;
   X³ is CR⁹, CR⁹R¹⁰, N, NR⁹, O, S, C═O, C═S, or a carbon atom connected to Q¹;
   X⁴ is CR¹¹, CR¹¹R¹², N, NR¹¹, O, S, C═O, C═S, or a carbon atom connected to Q¹; —X⁵ is CR¹³, N, or NR¹³;
wherein at least one of X¹, X², X³, and X⁴ is a carbon atom connected to Q¹;
   A is a linking group;
   Q¹ is C═O, C═S, C═CR¹⁴R¹⁵, C═NR¹⁴, alkylene, alkenylene, or alkynylene, each of which is independently substituted or unsubstituted, or a bond;
   m is 1, 2, 3, or 4;
   Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, and wherein each R¹ may be further substituted with the substituent -L-B_{c} or hydrogen;
   R³ is H;
   R⁴ is heterocyclyl substituted at least with halo-; and may be further substituted with the substituent -L-B_{c};
   each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
   each R¹⁹ and R²⁰ is C(O)R²³, —C(O)OR²³, —C(O)NR²³R²⁴, —OR², —SR², —NR²³R²⁴, —NR²C(O)R²⁴, —OC(O)R², alkyl, alkenyl, alkynyl, aryl, heteroaryl, or heterocyclyl, each of which is independently substituted or unsubstituted, or hydrogen or halogen;
   each R²¹ and R²² is independently alkyl, alkenyl, alkynyl, aryl, heteroaryl, or heterocyclyl, each of which is independently substituted or unsubstituted, or hydrogen; and
   each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

Another embodiment of the present invention is a bi-modal compound of the present invention suitable to target and modify a P53 protein with the IPPM and derived from PCT Application WO2021087096, with a selectively identified linkage to a Boron component of the present invention, wherein the target protein-binding moiety is derived, for example, from the compound of the formula:

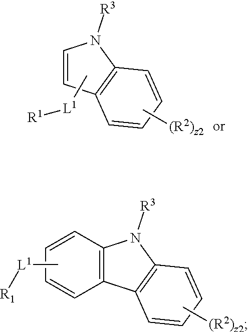

wherein
   L¹ is L;
   R¹ is B_{c}; R² is independently halogen, —CX²₃, —CHX²₂, —CH₂X², —OCX²₃, —OCH₂X², 7 —OCHX²2, —CN, —SOn2R^{2D}, —SOv2NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, 9 —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, 10 —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
   R^{2A}, R^{2B}, R^{2C}, and R^{2D} are independently hydrogen, —CCl₃, —CBr₃, —CF₃, —Cl₃, 17 —CHCl₂, —CHBr₂, —CHF₂, —CHI₂, —CH₂Cl, —CH₂Br, —CH₂F, —CH₂I, —CN, —OH, —NH₂, —COOH, 18 —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted
   heterocycloalkyl, substituted or unsubstituted aryl, or substituted or unsubstituted heteroaryl;
   R^{2A} and R^{2B} substituents bonded to the same nitrogen atom may optionally be joined to form a substituted or unsubstituted heterocycloalkyl or substituted or unsubstituted heteroaryl;
   X² is independently —F, —Cl, —Br, or —I;
   n2 is independently an integer from 0 to 4;
   m2 and v2 are independently 1 or 2;
   z2 is an integer from 0 to 7;
   R^{2A} and R^{2B} taken together or independently may be further substituted with the substituent -L-B_{c}; and
   R³ is a covalent cysteine modifier moiety.

In certain embodiments, the compound has the formula:

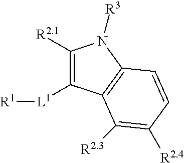

wherein R^{2.1}, R^{2.3}, and R^{2.4} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR²SO2R^{2D}, —NR^{2A}C(O)R^{2C}, —NR^{2A}C(O)OR^{2C}, —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, may be further substituted with the substituent -L-B_{c}.

In certain embodiments, R^{2.1} is hydrogen.

In certain embodiments, R^{2.4} is hydrogen.

In certain embodiments R^{2.3} is hydrogen, —NH₂, —NHCH₃, —N(CH3)

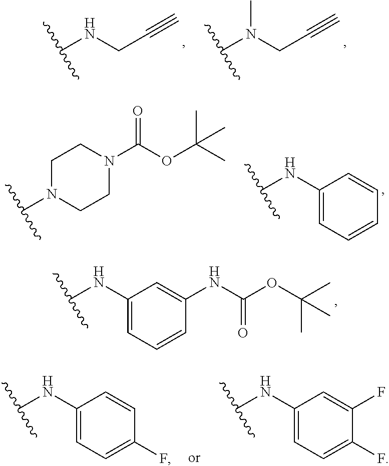

In certain embodiments, the compound has the formula:

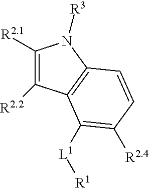

wherein
   R^{2.1}, R^{2.2}, and R^{2.4}, are independently hydrogen, halogen, —CX²₃, CHX²₂, —CH2X², —OCX²3, —OCH2X², —OCHX²2, —CN, —SOn2R^{2D}, —SOv2NR^{2A}R^{2B}, —NR^{2C}NR^{2A}R^{2B}, 6 —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R², 7 —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR², 8 —NR^{2A}OR^{2C}, —SF5, —N3, —NS(O)F2, —NS(O)FNR^{2A}R^{2B}, substituted or unsubstituted alkyl, 9 substituted or unsubstituted heteroalkyl, substituted or unsubstituted cycloalkyl, substituted or 10 unsubstituted heterocycloalkyl, substituted or unsubstituted aryl, or substituted or unsubstituted heteroaryl, which may be further substituted with the substituent -L-B_{c}.

In certain embodiments, R^{2.1} is hydrogen.

In certain embodiments, R^{2.4} is hydrogen.

In certain embodiments, R^{2.2} is —C(O)H, —CH₂C(O)H, —CH₂C(O)OH, —C(O)OH, —CH₂NHCH₃, —CH₂NH₂, or —CH₂N(CH₃)₂.

In certain embodiments, the compound has the formula:

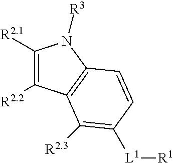

wherein
R^{2.1}, R^{2.2}, and R^{2.4}, are independently hydrogen, halogen, —CX²₃, —CHX²₂, —CH₂X², —OCX²3, —OCH2X², —OCHX²2, —CN, —SOn2R^{2D}, —SOv2NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO2R^{2D}, —NR^{2A}C(O)R^{2C}, —NR^{2A}C(O)OR^{2C}, —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, which may be further substituted with the substituent -L-B_{c}.

In certain embodiments, R^{2.1} is hydrogen.

In certain embodiments, R^{2.2} is hydrogen, —C(O)H, —CH₂C(O)H, —CH₂C(O)OH, —C(O)OH, —CH₂NHCH₃, —CH₂NH₂, or —CH₂N(CH₃)₂.

In certain embodiments, R^{2.3} is
hydrogen, —NH2, —NHCH3, —N(CH3)3

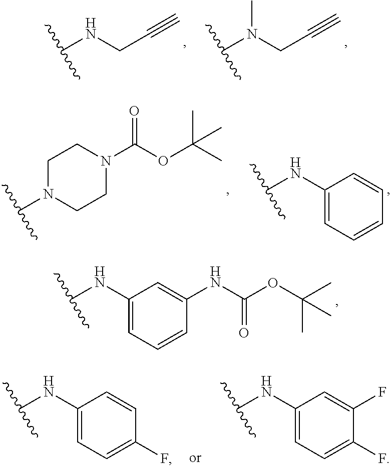

wherein
R^{2.2}, R^{2.3}, and R^{2.4} are independently hydrogen, halogen, —CX², —CHX²₂, —CH₂X², —OCX²₃, —OCH₂X², —OCHX²₂, —CN, —SOn2R^{2D}, —SOv2NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, which may be further substituted with the substituent -L-B_{c}.

In certain embodiments, the compound has the formula:

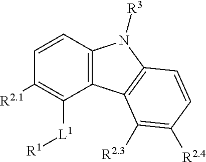

wherein
   R^{2.1}, R^{2.3}, and R^{2.4} are independently hydrogen, halogen, —CX²3, —CHX²2, 5 —CH₂X², —OCX²₃, —OCH₂X², —OCHX²₂, —CN, —SOn2R^{2D}, —SOᵥ₂NR^{2A}R^{2B}, —NR^{2C}NR^{2A}R^{2B}, 6 —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, 7 —C(O)—OR^{2C}, —C(O)NR^{2A}R^{2B}, —OR^{2D}, —NR^{2A}SO2R^{2D}, —NR^{2A}C(O)R^{2C}, —NR^{2A}C(O)OR^{2C}, 8 —NR^{2A}OR^{2C}, —SF₅, —N₃, —NS(O)F2, —NS(O)FNR^{2A}R^{2B}, substituted or unsubstituted alkyl,
   substituted or unsubstituted heteroalkyl, substituted or unsubstituted cycloalkyl, substituted or unsubstituted heterocycloalkyl, substituted or unsubstituted aryl, or substituted or unsubstituted heteroaryl which may be further substituted with the substituent -L-B_{c}.

In certain embodiments, the compound has the formula:

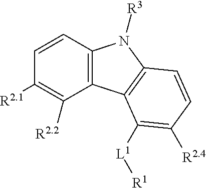

wherein
   4 R^{2.1}, R^{2.2}, and R^{2.4} are independently hydrogen, halogen, —CX²3, —CHX²2, 5 —CH2X², —OCX²3, —OCH₂X², —OCHX²2, —CN, —SOn2R^{2D}, —SOv2NR^{2A}R^{2B}, —NR^{2C}NR^{2A}R^{2B}, 6 —ONR^{2A}R^{2B}, —NHC(O)NR^{2C}NR^{2A}R^{2B}, —NHC(O)NR^{2A}R^{2B}, —N(O)m2, —NR^{2A}R^{2B}, —C(O)R^{2C}, 7 —C(O)—OR^{2C}, —C(O)NR^{2A}R^{2B}, —OR^{2D}, —NR^{2A}SO2R^{2D}, —NR^{2A}C(O)R^{2C}, —NR^{2A}C(O)OR^{2C}, 8 —NR^{2A}OR^{2C}, —SF5, —N3, —NS(O)F2, —NS(O)FNR^{2A}R^{2B}, substituted or unsubstituted alkyl,
   substituted or unsubstituted heteroalkyl, substituted or unsubstituted cycloalkyl, substituted or
   unsubstituted heterocycloalkyl, substituted or unsubstituted aryl, or substituted or unsubstituted heteroaryl, which may be further substituted with the substituent -L-B_{c}.

In certain embodiments, the compound has the formula:

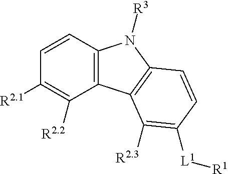

wherein
   R^{2.1}, R^{2.2}, and R^{2.3} are independently hydrogen, halogen, —CX²₃, —CHX²₂, 5 —CH2X², —OCX²3, —OCH2X², —OCHX²2, —CN, —SOn2R^{2D}, —SOv2NR^{2A}R^{2B}, —NR^{2C}NR^{2A}R^{2B}, 6 —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R², 7 —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C},
   —NR^{2A}OR^{2C}, —SF₅, —N₃, —NS(O)F₂, —NS(O)FNR^{2A}R^{2B}, substituted or unsubstituted alkyl,
   substituted or unsubstituted heteroalkyl, substituted or unsubstituted cycloalkyl, substituted or
   unsubstituted heterocycloalkyl, substituted or unsubstituted aryl, or substituted or unsubstituted heteroaryl, which may be further substituted with the substituent -L-B_{c}.

In certain embodiments, R^{2.1} is —C(O)H, —CH₂C(O)H, —CH₂C(O)OH, —C(O)OH, —CH₂NHCH₃, —CH₂NH₂, or —CH₂N(CH₃)₂.

In certain embodiments, R³ is

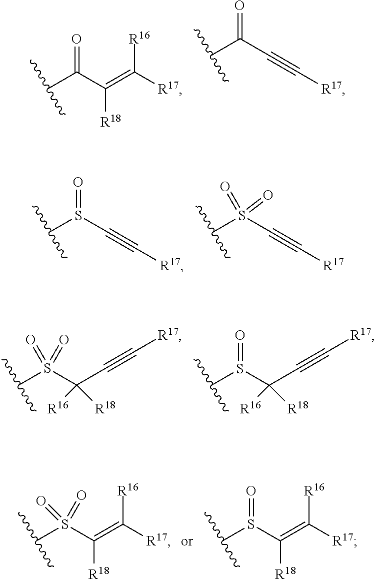

and
R¹⁶, R¹⁷, and R¹⁸ are independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In certain embodiments, R³ is

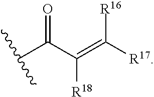

In certain embodiments, R¹⁶ is hydrogen, and R¹⁸ is hydrogen.

In certain embodiments, R¹⁶ is hydrogen, R¹⁷ is hydrogen, 2 and R¹⁶ is unsubstituted methyl.

In certain embodiments, R¹⁶ is hydrogen, R¹⁷ is hydrogen, 2 and R¹⁸ is —CN.

In certain embodiments, the compound has the formula:

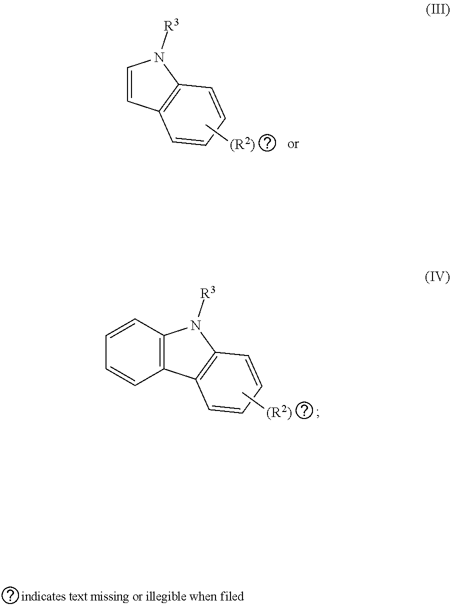

wherein
   R² is independently halogen, —CX²3, —CHX²2, —CH₂X², —OCX²3, —OCH₂X², —OCHX²2, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl, which may be further substituted with the substituent -L-B_{c};
   R^{2A}, R^{2B}, R^{2C}, and R^{2D} are independently hydrogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂₁, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl, and is further substituted with the substituent -L-B_{c};
   X² is independently —F, —Cl, —Br, or —I;
   n2 is independently an integer from 0 to 4;
   m2 and v2 are independently 1 or 2;
   z2a is an integer from 0 to 8; and
   R³ is a covalent cysteine modifier moiety.

In certain embodiments, the compound has the formula:

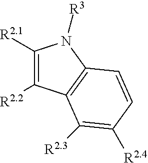

wherein
   R^{2.1}, R^{2.2}, R^{2.3}, and R^{2.4} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR², —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and is further substituted with the substituent -L-B_{c};
   In certain embodiments, R^{2.1} is hydrogen.

In certain embodiments, R^{2.4} is hydrogen.

In certain embodiments, R^{2.2} is —C(O)H, —CH₂C(O)H, —CH₂C(O)OH, —C(O)OH, —CH₂NHCH₃, —CH₂NH₂, or —CH₂N(CH₃)₂.

In certain embodiments, R^{2.3} is hydrogen, —NH₂, —NHCH₃, —N(CH₃)₃,

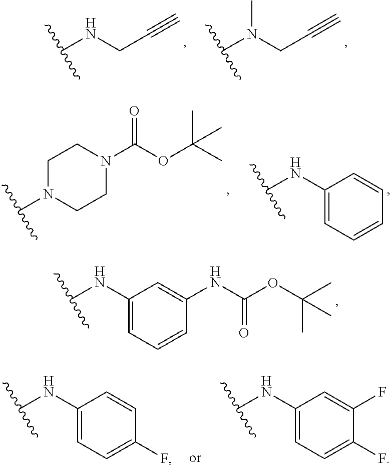

In certain embodiments, the compound has the formula:

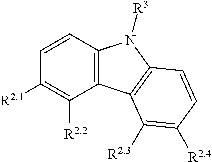

wherein
R^{2.1}, R²², R^{2.3}, and R^{2.4} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R², —C(O)—OR^{2C}, —C(O)NR^{2A}R^{2B}, —OR^{2D}, —NR^{2A}SO2R^{2D}, —NR²AC(O)R², —NR²AC(O)OR², —NR^{2A}OR^{2C}, —SF₅, —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and is further substituted with the substituent -L-B_{c}.

In certain embodiments, R^{2.1} is —C(O)H, —CH₂C(O)H, —CH₂C(O)OH, —C(O)OH, —CH₂NHCH₃, —CH₂NH₂, or —CH₂N(CH₃)₂.

In certain embodiments, R³ is

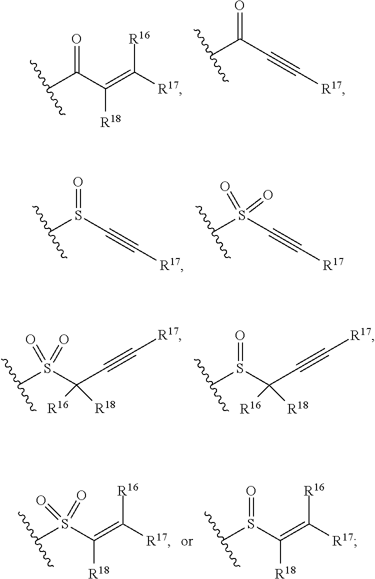

and
—CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl,
substituted or unsubstituted cycloalkyl, substituted or unsubstituted heterocycloalkyl,
substituted or unsubstituted aryl, or substituted or unsubstituted heteroaryl

In certain embodiments, R³ is

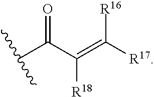

In certain embodiments, R¹⁶ is hydrogen, R¹⁷ is hydrogen, and R¹⁸ is hydrogen.

In certain embodiments, R¹⁶ is hydrogen, R¹⁷ is hydrogen, and R¹⁸ is unsubstituted methyl.

In certain embodiments, R¹⁶ is hydrogen, R¹⁷ is hydrogen, and R¹⁸ is —CN.

ii. PI3K Pathway

Another embodiment of the present invention is a bi-modal compound of the present invention suitable to target and modify a PI3K protein with the IPPM and derived from PCT Application WO2022265993, with a selectively identified linkage to a Boron component of the present invention, wherein the target protein-binding moiety is derived, for example, from the compound of the formula:

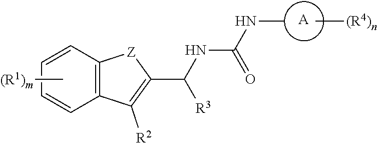

or a pharmaceutically acceptable salt thereof, wherein:
   Z is O or NR^{x};
   R^{x} is hydrogen, C₁-C₆ alkyl, or C₃-C₆ cycloalkyl;
   each R¹ is independently selected from halogen, hydroxyl, cyano, C₁-C₆ alkyl optionally substituted with hydroxyl, and C₃-C₆ cycloalkyl;
   m is 0, 1, 2, or 3;
   R² is halogen, hydroxyl, C₁-C₆ alkyl optionally substituted with hydroxyl, C₁-C₆ haloalkyl, C₃-C₆ cycloalkyl optionally substituted with 1 or 2 fluoro;
   R³ is a C₁-C₆ alkyl, a C₁-C₆ haloalkyl, or a C₃-C₆ cycloalkyl optionally substituted with 1 or 2 substituents independently selected from fluoro and C₁-C₆ alkyl;
   Ring A is a 6-10 membered aryl, a C₃-C₈ cycloalkyl, a 5-10 membered heteroaryl, or a 4-10 membered heterocyclyl;
   each R⁴ is independently selected from the group consisting of:
      (i) halogen,
      (ii) C₁-C₆ alkyl optionally substituted with 1 or 2 hydroxyl or —NR^{A}R^{B},
      (iii) C1-C₆ alkoxy optionally substituted with 1-2 substituents independently selected from hydroxyl and C₃-C₆ cycloalkyl,
      (iv) C₁-C₆ haloalkyl,
      (v) hydroxyl,
      (vi) cyano,
      (vii) —CO₂H,
      (viii) —NR^{A}R^{B},
      (ix) ═NR^{A2},
      (x) —C(═O)NR^{C}R^{D},
      (xi) —SO²(NR^{E}R^{F}),
      (xii) —SO₂(C₁-C₆ alkyl),
      (xiii) —S(═O)(═NH)(C₁-C₆ alkyl),
      (xiv) —C(═O)(C₁-C₆ alkyl),
      (xv) —CO₂(C₁-C₆ alkyl),
      (xvi) 5-6 membered heteroaryl optionally substituted with C₁-C₆ alkyl,
      (xvii) 3-9 membered heterocyclyl optionally substituted with 1 or 2 independently selected R^{G}, and
      (xviii) 3-6 membered cycloalkyl optionally substituted with 1 or 2 independently selected R^{G},
wherein R⁴ is further substituted with the substituent -L-B_{c},
   n is 0, 1, or 2;
   each R^{A}, R^{A}, R^{B}, R^{B1}, R^{C}, R^{C1}, R^{D}, R^{D1}, R^{E}, and R^{F} is independently
      (i) hydrogen,
      (ii) hydroxyl,
      (iii) 4-6 membered heterocyclyl,
      (iv) C₁-C₆ haloalkyl,
      (v) —C(═O)(C₁-C₆ alkyl),
      (vi) —C(═O)O(C₁-C₆ alkyl),
      (vii) —SO₂(C₁-C₆ alkyl),
      (viii) 3-6 membered cycloalkyl optionally substituted with hydroxyl, or
      (ix) C₁-C₆ alkyl optionally substituted with 1-2 substituents independently selected from hydroxyl, —C(═O)NR^{B2}R^{C2}, 5-6 membered heteroaryl, 3-6 membered cycloalkyl, —SO₂(C₁-C₆ alkyl), —CO₂H, and —SO₂(NH₂); or
   R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C₁-C₆ alkyl), —CO₂H, C₁-C₆ alkyl optionally substituted with hydroxyl, C₁-C₆ alkoxy, and C₁-C₆ haloalkoxy;
   each R^{A2}, R^{B2}, and R^{C2} is independently hydrogen or C₁-C₆ alkyl;
   each R^{G} is independently selected from the group consisting of: fluoro, cyano, hydroxyl, C₁-C₆ alkyl optionally substituted with hydroxyl, C₁-C₆ alkoxy, —NR^{A1}R^{B1}, ═NR^{A2}, —C(═O)NR^{C1}R^{D1}, —CO²(C₁-C₆ alkyl), C₁-C₆ haloalkyl, C₃-C₆ cycloalkyl, C₁-C₆ haloalkoxy, —SO₂(C₁-C₆ alkyl), and —CO₂H.

In certain embodiments, m is 1.

In certain embodiments, m is 2.

In certain embodiments, R¹ is halogen.

In certain embodiments, each R¹ is selected from fluoro and chloro.

In certain embodiments, each R¹ is fluoro.

In certain embodiments, one R¹ is cyano.

In certain embodiments, one R¹ is C₁-C₆ alkyl or C₃-C₆ cycloalkyl.

In certain embodiments, m is 0.

In certain embodiments, R² is a C₁-C₆ alkyl.

In certain embodiments, R² is methyl.

In certain embodiments, R² is a C₁-C₆ haloalkyl.

In certain embodiments, R² is difluoromethyl.

In certain embodiments, R² is trifluoromethyl.

In certain embodiments, R² is halogen.

In certain embodiments, R² is C₃-C₆ cycloalkyl optionally substituted with 1 or 2 fluoro.

In certain embodiments, R³ is a C₁-C₆ haloalkyl.

In certain embodiments, R² is difluoromethyl.

In certain embodiments, R² is trifluoromethyl.

In certain embodiments, R³ is a C₁-C₆ alkyl.

In certain embodiments, R³ is C₃-C₆ cycloalkyl optionally substituted with 1 or 2 substituents independently selected from fluoro and C₁-C₆ alkyl.

In certain embodiments, Ring A is a 5-10 membered heteroaryl.

In certain embodiments, Ring A is a 5-6 membered heteroaryl.

In certain embodiments, Ring A is pyrimidinyl, pyridyl, thiazolyl, thiophenyl, or pyrazolyl.

In certain embodiments, Ring A is pyrimidinyl.

In certain embodiments, Ring A is pyrdyl.

In certain embodiments, Ring A is thiazolyl.

In certain embodiments, Ring A is thiophenyl.

In certain embodiments, Ring A is pyrazolyl.

In certain embodiments, Ring A is a 9-10 membered heteroaryl.

In certain embodiments, Ring A is benzimidazolyl, indazolyl, indolyl, quinazolone, isobenzofuranonyl, isoindolinonyl, or imidazo[1,2-a]pyridinyl.

In certain embodiments, Ring A is benzimidazolyl.

In certain embodiments, Ring A is indazolyl.

In certain embodiments, Ring A is indolyl.

In certain embodiments, Ring A is quinazolone.

In certain embodiments, Ring A is isobenzofuranonyl.

In certain embodiments, Ring A is isoindolinonyl.

In certain embodiments, Ring A is imidazo[1,2-a]pyridinyl.

In certain embodiments, Ring A is phenyl.

In certain embodiments, Ring A is a C₃-C₈ cycloalkyl.

In certain embodiments, Ring A is a 4-10 membered heterocyclyl.

In certain embodiments, Ring A is a 4-6 membered heterocyclyl.

In certain embodiments, n is 1.

In certain embodiments, n is 2.

In certain embodiments, one R⁴ is an unsubstituted C₁-C₆ alkyl and is further substituted with the substituent -L-B_{c}.

In certain embodiments, one R⁴ is C₁-C₆ alkoxy optionally substituted with 1-2 substituents independently selected from hydroxyl and C₃-C₆ cycloalkyl, and is further substituted with the substituent -L-B_{c}.

In certain embodiments, one R⁴ is C₁-C₆ haloalkyl and is further substituted with the substituent -L-B_{c}.

The compound of any one of claims 1-45, wherein one R⁴ is hydroxyl, cyano, —CO₂H, halogen, or C₁-C₆ alkyl substituted with 1-2 hydroxyl or —NR^{A}R^{B}, and is further substituted with the substituent -L-B_{c}

In certain embodiments, one R⁴ is —NR^{A}R^{B}, —C(═O)NR^{C}R^{D}, —SO₂(NR^{E}R^{F}), —SO₂(C₁-C₆ alkyl), —S(═O)(═NH)(C₁-C₆ alkyl), —C(═O)(C₁-C₆ alkyl), or —CO₂(C₁-C₆ alkyl), and is further substituted with the substituent -L-B_{c}.

In certain embodiments, one R⁴ is 5-6 membered heteroaryl optionally substituted with C₁-C₆ alkyl, and is further substituted with the substituent -L-B_{c}.

In certain embodiments, one R⁴ is 3-9 membered heterocyclyl optionally substituted with 1 or 2 independently selected RG, and is further substituted with the substituent -L-B_{c}.

In certain embodiments, Z is 0.

In certain embodiments, Z is NR^{x}

In certain embodiments, the compound comprises the following formula:

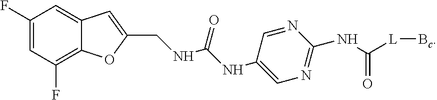

In certain embodiments, L-B_{C} is selected from the group consisting of:

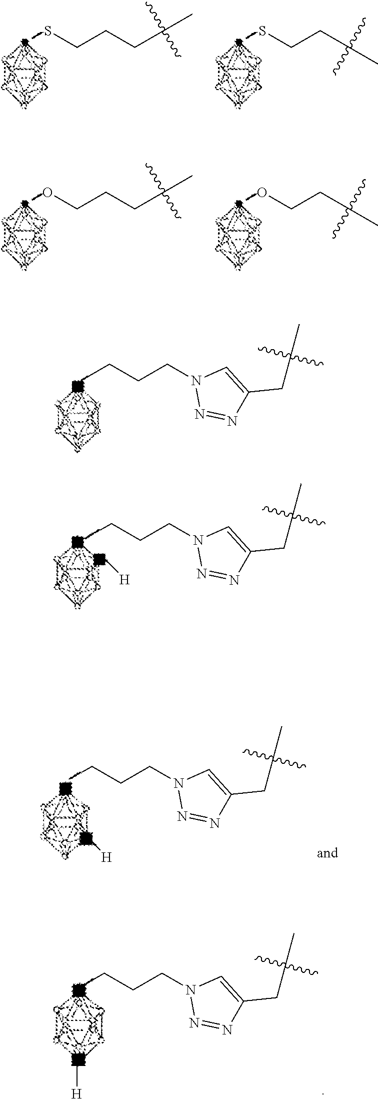

For example, in certain embodiments, the compound comprises the following formula:

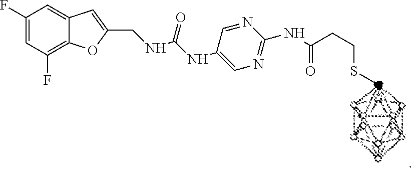 [SMILES: CSCCC(=O)Nc1ncc(NC(=O)NCc2cc3cc(F)cc(F)c3o2)cn1]

Another embodiment of the present invention is a bi-modal compound of the present invention suitable to target and modify a PI3K protein with the IPPM and derived from PCT Application WO2021202964, with a selectively identified linkage to a Boron component of the present invention, wherein the target protein-binding moiety is derived, for example, from the compound of the formula:

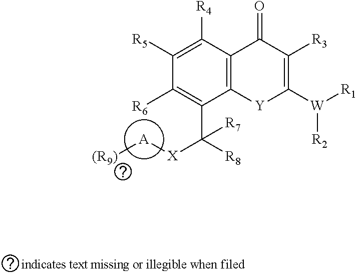

or a prodrug, solvate, enantiomer, stereoisomer, tautomer, or pharmaceutically acceptable salt thereof, wherein:
   X is —NR¹²— or —O—;
   Y is —C(R₁₁)₂—, —O—, —NR₁₁—, or —S—;
   WR₁R₂ is a group of the formula:

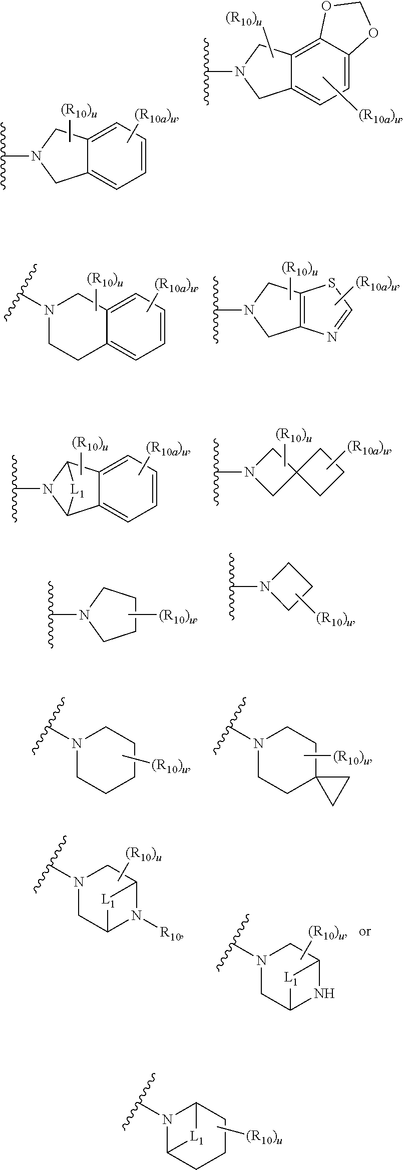

R₁₀ at each occurrence is independently oxo, halogen, —CN, C₁-C₆ alkyl, C₂-C₆ alkenyl, C₂-C₆ alkynyl, C₁-C₆ haloalkyl, C₁-C₆ alkoxy, —(CH₂)ₙ—OR₁₂, —(CH₂)ₙ—N(R₁₂)₂, —(CH₂)ₙ—C(O)R₁₂, —(CH₂)ₙ—C(O)OR₁₂, —(CH₂)ₙ—C(O)N(R₁₂)₂, —(CH₂)ₙ—SO₂R₁₂, —(CH₂)ₙ—O—(CH₂CH₂—O)ᵣR₁₃, C₃-C₁₀ cycloalkyl, heterocycle, —(CH₂)ₙ-aryl, or heteroaryl, wherein the cycloalkyl, heterocycle, aryl, and heteroaryl is optionally substituted with halogen, C₁-C₆ alkyl, C₂-C₆ alkenyl, C₂-C₆ alkynyl, C₁-C₆ haloalkyl, C₁-C₆ alkoxy, C₁-C₆ haloalkoxy, or —(CH₂)ₙ—SO₂R₁₂, which may be further substituted with the substituent -L-B_{c};
   R₁₀ₐ at each occurrence is independently halogen, —CN, C₁-C₆ alkyl, C₁-C₆ haloalkyl, C₁-C₆ alkoxy, or —(CH₂)ₙ—OR₁₂;
   L₁- is —(CH₂)— or —(CH₂)₂—;
   u at each occurrence is independently 0, 1, 2, 3 or 4;
each R₃, R₄, R₅, and R₆ is independently H, halogen, —CN, C₁-C₆ alkyl, C₂-C₆ alkenyl, C₂-C₆ alkynyl, C₁-C₆ haloalkyl, C₁-C₆ alkoxy, —(CH₂)ₘ—R₁₂, —(CH₂)ₘ—R₁₂, —CH₂)ₘ—N(R₁₂)₂, —(CH₂)ₘ—C(O)R₁₂, —(CH₂)ₘ—C(O)OR₁₂, —(CH₂)ₘ—C(O)N(R₁₂)₂, C₃-C₁₀ cycloalkyl, aryl, heterocycle comprising 1-4 heteroatoms selected from o, N, and S, or heteroaryl comprising 1-4 heteroatoms selected from O, N, and S, which may be further substituted with the substituent -L-B_{c};
   each R₇ and R₅ is independently H, halogen, —CN, C₁-C₆ alkyl, C₂-C₆ alkenyl, C₂-C₆ alkynyl, C₁-C₆ haloalkyl, or C₁-C₆alkoxy;
   at least one R₉ is —C(O)OR₁₂ and each of the remaining R₉ at each occurrence is independently oxo, ═NR₁₁, halogen, —CN, —NO₂, C₁-C₆ alkyl, C₂-C₆ alkenyl, C₂-C₆ alkynyl, C₁-C₆ haloalkyl, C₁-C₆ alkoxy, —(CH₂)ₘ—N(R₁₂)₂, —(CH₂)ₘ—OR₁₂, —(CH₂)ₘ—CR₁₃(OH)—R₁₂, —(CH₂)ₘ—C(O)R₁₂, —(CH₂)ₘ—C(O)OR₁₂, —(CH₂)ₘ—C(O)N(R₁₂)₂, —(CH₂)ₘ—C(O)N(OH)R₁₂, —(CH₂)ₘ—SO₂R₁₂, —(CH₂)ₘ—SO₂—OR₁₂, —(CH₂)ₘ—SO₂N(R₁₂)₂, —(CH₂)ₘ—P(O)(OR₁₂)₂, —(CH₂)ₘ—P(O)(R₁₂)₂, —(CH₂)ₘ—P(O)(OR₁₃)R₁₂, —(CH₂)ₘ—B(OH)₂, —(CH₂)ₘ—B(R₁₂)₂, —(CH₂)ₘ—O—(CH₂CH₂—O)ᵣR₁₃, —(CH₂)ₘ—NR₁₂—(CH₂CH₂—O)ᵣR₁₃, —(CH₂)ₘ—C(O)—(CH₂CH₂—O)ᵣR₁₃, —(CH₂)ₘ—C(O)O—(CH₂CH₂—O)ᵣR₁₃, —(CH₂)ₘ—C(O)NR₁₂—(CH₂CH₂—O)ᵣR₁₃, —(CH₂)ₘ—C(O)—NR₁₂—SO₂R₁₃, —(CH₂)ₘ—SO₂NR₁₂—C(O)R₁₃, —(CH₂)ₘ—S(O)(NR₁₂)—R₁₃, C₃-C₁₀ cycloalkyl, aryl, heterocycle comprising 1-4 heteroatoms selected from O, N, and S, or heteroaryl comprising 1-4 heteroatoms selected from N, O, and S, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, aryl, heterocycle, or heteroaryl is optionally substituted with one or more oxo, halogen, —CN, —OH, —NH₂, —NO₂, C₁-C₆ alkyl, C₂-C₆ alkenyl, C₂-C₆ alkynyl, C₁-C₆ haloalkyl, or C₁-C₆ alkoxy; or two R₉, together with the atoms to which they are attached form a C₃-C₁₀ cycloalkyl, an aryl, or a heterocycle comprising 1-4 heteroatoms selected from O, N, and S, wherein the cycloalkyl, aryl or heterocycle is optionally substituted with one or more oxo, halogen, —CN, —OH, —NH₂, ═NH, —NO₂, C₁-C₆ alkyl, C₂-C₆ alkenyl, C₂-C₆ alkynyl, C₁-C₆ haloalkyl, or C₁-C₆ alkoxy, which may be further substituted with the substituent -L-B_{c};
   R₁₁ is H, C₁-C₆ alkyl, C₂-C₆ alkenyl, or C₂-C₆ alkynyl;
   each R₁₂ and R₁₃ at each occurrence is independently H, C₁-C₆ alkyl, C₂-C₆ alkenyl, C₂-C₆ alkynyl, C₁-C₆ haloalkyl, C₁-C₆ alkoxy, —(CH₂)_{q}—O—C(O)—(CH₂)ᵣ—R₁₄, —(CH₂)_{q}—NH—C(O)—(CH₂)ᵣR₁₄, —(CH₂)_{q}—O—C(O)—(CH₂)ᵣOR₁₄, —(CH₂)_{q}—NH—C(O)—(CH₂)ᵣOR₁₄, —(CH₂)_{q}—O—(CH₂)ᵣR₁₄, —(CH₂)_{q}—NH—(CH₂)ᵣR₁₄, —(CH₂)_{q}—O—(CH₂)ᵣ OR₁₄, —(CH₂)_{q}—NH—(CH₂)ᵣOR₁₄, C₃-C₁₀ cycloalkyl, heterocycle comprising 1-4 heteroatoms selected from O, N, and S, —(CH₂)_{q}-aryl, or heteroaryl comprising 1-4 heteroatoms selected from N, O, and S, wherein the cycloalkyl, heterocycle, aryl, and heteroaryl are optionally substituted with one or more halogen, C₁-C₆ alkyl, C₁-C₆ haloalkyl, C₁-C₆ alkoxy, or C₁-C₆ haloalkoxy;
   Ring A is C₃-C₁₀ cycloalkyl, aryl, heterocycle comprising 1-4 heteroatoms selected from N, O, and S, or heteroaryl comprising 1-4 heteroatoms selected from N, O, and S;

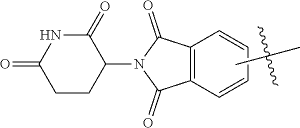

each n, m, q, or r, is independently at each occurrence 0, 1, 2, 3, 4, 5, or 6; and s is 1, 2, 3, 4, 5, or 6.
with the proviso that at least one of R₃, R₄, R₅, R₆, R₉, or R₁₀ are substituted with the substituent -L-B_{c}.

In certain embodiments, the compound has the formula:

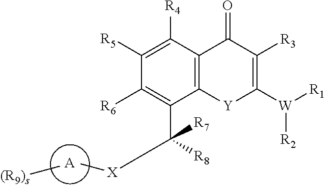

wherein
R₇ is halogen, —CN, C₁-C₆ alkyl, C₂-C₆ alkenyl, C₂-C₆ alkynyl, C₁-C₆ haloalkyl, or C₁-C₆ alkoxy; and R₈ is H.

In certain embodiments, X is —NR₁₂—.

In certain embodiments, Y is —O—

In certain embodiments, each R₃, R₄, R₅, and R₆ is independently H, halogen, —CN, C₁-C₆ alkyl, C₁-C₆ haloalkyl, C₁-C₆ alkoxy, —(CH₂)ₘ—OR₁₂, —(CH₂)ₘ—N(R₁₂)₂, —(CH₂)ₘ—C(O)R₁₂, C₃-C₁₀ cycloalkyl, aryl, heterocycle comprising 1-4 heteroatoms selected from O, N, and S, or heteroaryl comprising 1-4 heteroatoms selected from O, N, and S, which is further substituted with the substituent -L-B_{c}.

In certain embodiments, R₃ is hydrogen.

In certain embodiments, R₃ is —CN, C₁-C₆ alkyl, C₁-C₆ haloalkyl, —(CH₂)ₘ—OR₁₂, —(CH₂)ₘ—C(O)R₁₂, C₃-C₁₀ cycloalkyl, aryl or heteroaryl comprising 1-4 heteroatoms selected from O, N, and S.

In certain embodiments, R₃ is —CN or C₁-C₃ alkyl.

In certain embodiments, R₄ is H, halogen, C₁-C₆ alkyl or C₁-C₆ haloalkyl, which may be further substituted with the substituent -L-B_{c}.

In certain embodiments, R₅ is H, halogen, C₁-C₆ alkyl, C₁-C₆ haloalkyl, C₁-C₆ alkoxy, —(CH₂)ₘ—OR₁₂, —(CH₂)ₘ—N(R₁₂)₂, or heterocycle comprising 1-4 heteroatoms selected from O, N, and S, which may be further substituted with the substituent -L-B_{c}.

In certain embodiments, R₅ is H, halogen, methyl or trifluorom ethyl.

In certain embodiments, each R₆ is H.

In certain embodiments, R₇ is —CN, C₁-C₆ alkyl or C₁-C₆ haloalkyl and R₅ is H.

In certain embodiments, R₇ is C₁-C₃ alkyl and R₆ is H.

In certain embodiments, Ring A is a group of the formula:

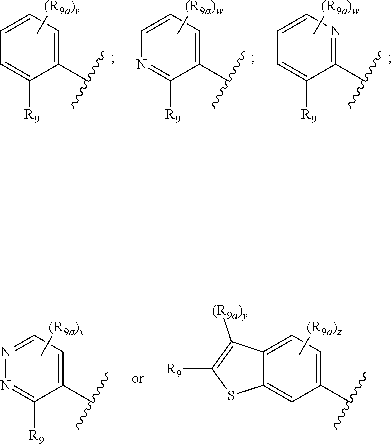

wherein R₉ is —C(O)OH, R₉ₐ is halogen, C₁-C₃ alkyl, C₁-C₃ haloalkyl, C₁-C₃ alkoxy, or C₃-C₅ cycloalkyl, v is 0, 1, or 2, w is 0, 1, or 2, x is 0 or 1, y is 0 or 1, and z is 0, 1, or 2, wherein R₉ is further substituted with the substituent -L-B_{c};

In certain embodiments, Ring A is a group of the formula:

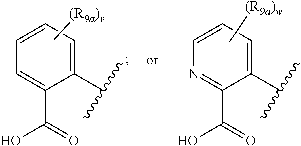

wherein R₉ₐ is halogen or trifluorom ethyl; v is 0 or 1; and w is 0 or 1, R₉ wherein R₉ is further substituted with the substituent -L-B_{c}.

In certain embodiments, the compound comprises the following formula:

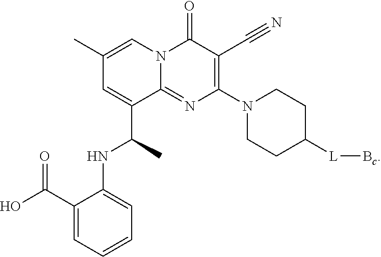

In certain embodiments, L-B_{C} is selected from the group consisting of:

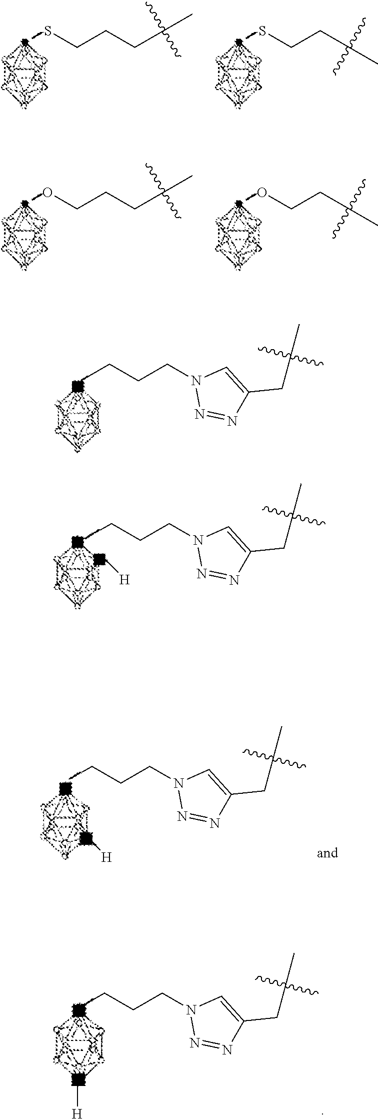

iii. KRAS Pathway

Another embodiment of the present invention is a bi-modal compound of the present invention suitable to target and modify a KRAS protein with the IPPM and derived from PCT Application WO2021118877, with a selectively identified linkage to a Boron component of the present invention, wherein the target protein-binding moiety is derived, for example, from the compound of the formula:

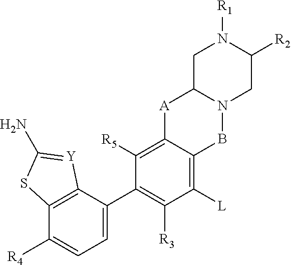

wherein:
   A is —OCH₂—, —N(R₆)CH₂, —OCH₂CH₂—, —N(R₆)CH₂CH₂—, —CH₂OCH₂—, or —CH₂N(R₆)CH₂—;
   B is —CH₂— or —C(O)—;
   Y is —C(CN)— or —N—;
   R₁ is —CN, —C(O)C≡CR₈, H, or a group of the formula

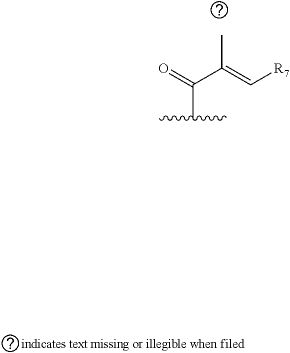

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
L is a linking structure described herein throughout and substituted with B_{C} forming the substituent -L-B_{c};
R₇ is H, halogen, —NR₁₁R₁₂, —CH₂NR₁₁R₁₂, —C₁₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C_{10.4} alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁-4 alkyl;
R₁₁ and R₁₂ are each independently H, —C₁₋₄ alkyl, or —C₁₋₄ heteroalkyl, wherein R₁₁ and R₁₂ may combine to form a cycloheteroalkyl; and
R₁₃ is independently at each occurrence —N—C₁₋₄ alkyl, H,
or a pharmaceutically acceptable salt thereof.

In certain embodiments, A is —OCH₂CH₂—.

In certain embodiments, B is —C(O)—.

In certain embodiments, Y is —C(CN)—.

In certain embodiments, Y is —N—.

In certain embodiments, R₁ is a group of the formula

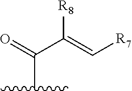

and wherein R₇ is H, F, Cl, methyl, ethoxy, ethyl, isopropyl, or cyclopropyl.

In certain embodiments, R₁ is a group of the formula

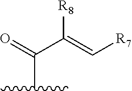

and wherein R₉ is H, F, Cl, —CHF₂, —CF₃, or —CH₂OH.

In certain embodiments, R₁ is —CN, —C(O)C≡CR₈, or H.

In certain embodiments, R₂ is H or methyl.

In certain embodiments, R₃ is H, F, Cl, methyl, methoxy, ethyl, isopropyl, or cyclopropyl.

In certain embodiments, R₄ is H, F, or Cl.

In certain embodiments, R₅ is H, —CHF₂, —CH₂F, —CH₂OH, or —CH₂OCH₃.

In certain embodiments, the compound has the formula:

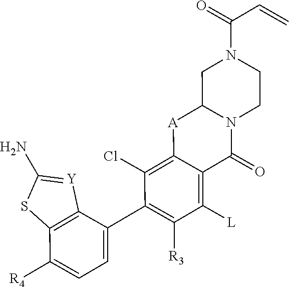

wherein:
   A is —OCH₂— or —OCH₂CH₂—;
   Y is C(CN) or N;
   R₃ is Cl or F;
   R₄ is H or F when Y is C(CN); and
   R₄ is F when Y is N,
   or a pharmaceutically acceptable salt thereof.
   L is further comprised of the substituent -L-B_{c}.

In certain embodiments, A is

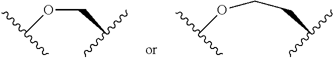

In certain embodiments, the compound has the formula:

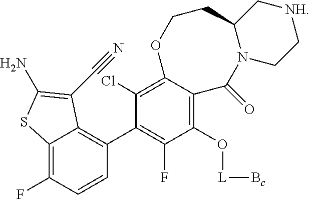

In certain embodiments, the compound has the formula:

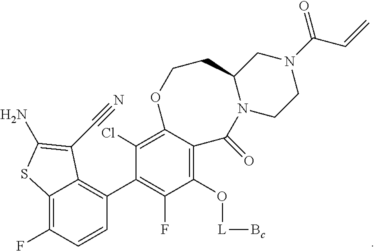

In certain embodiments, L-B_{C} is selected from the group consisting of:

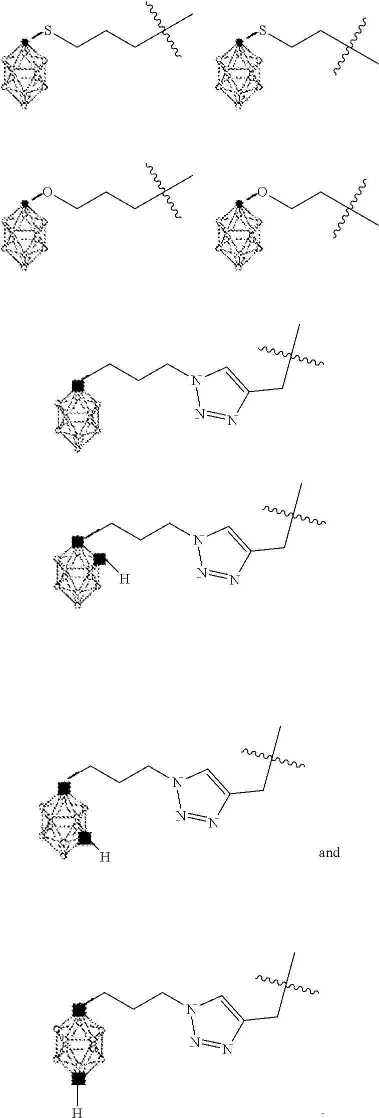

In certain embodiments, the compound has the formula is selected from:

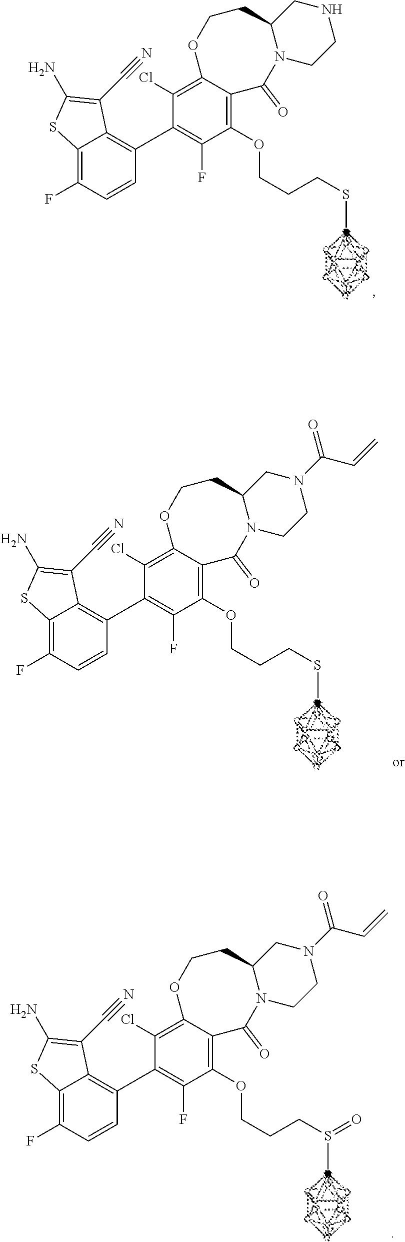 [SMILES: CSCCCOc1c(F)c(-c2ccc(F)c3sc(N)c(C#N)c23)c(Cl)c2c1C(=O)N1CCNC[C@@H]1CCO2 ; C=CC(=O)N1CCN2C(=O)c3c(OCCCSC)c(F)c(-c4ccc(F)c5sc(N)c(C#N)c45)c(Cl)c3OCC[C@H]2C1 ; C=CC(=O)N1CCN2C(=O)c3c(OCCCS(C)=O)c(F)c(-c4ccc(F)c5sc(N)c(C#N)c45)c(Cl)c3OCC[C@H]2C1]

Another embodiment of the present invention is a bi-modal compound of the present invention suitable to target and modify a KRAS protein with the IPPM and derived from US Published Application US20240025907, with a selectively identified linkage to a Boron component of the present invention, wherein the target protein-binding moiety is derived, for example, from the compound of the formula:

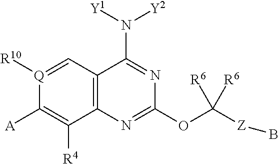

or a pharmaceutically acceptable salt thereof,
wherein:
   A is aryl or heteroaryl, wherein the aryl or the heteroaryl is optionally substituted with 1-4 R¹;
   B is:

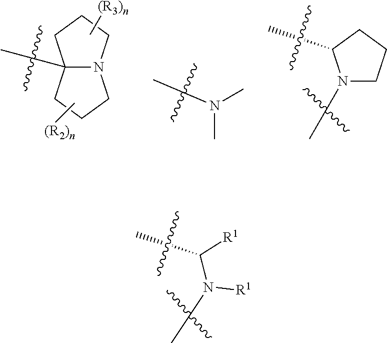

substituted with the substituent -L-B_{c};
Y¹ is hydrogen, hydroxy, halogen, Z—SO₂—NH₂, Z—OH, C₁-C₄ alkyl, Z—C3-C6 cycloalkyl optionally substituted with 1-4 R⁹, Z-heteroaryl optionally substituted with 1-4 R⁸, Z-aryl optionally substituted with 1-4 R⁸, Z—C(O)—NH₂, and Z-heterocycle optionally substituted with 1-2 oxo (═O) or oxo-containing substituent, and optionally further substituted with 1-2 R₈, Z is a bond, —C₁-C₄ alkyl-, —NH—, —N(C₁-C₃ alkyl)- or cyclopropyl-CH₂—;
Y² is hydrogen or C₁-C₄ alkyl;
or Y¹ and Y² join to form a monocyclic or bicyclic substituted or unsubstituted heterocyclyl, or:
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, deuterium, hydroxy, halogen, C1-C3 alkyl, ═CH₂, ═CH(halogen), =C(halogen)₂, C1-C3 cyanoalkyl, C1-C3 hydroxyalkyl, HC(═O)—, —OC(O)N(R⁵)₂, —CO₂R⁵, or —CO₂N(R⁵)₂;
each R³ is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —COC(O)N(R⁵)₂, —CO₂R⁵, —CO₂N(R⁵)₂, or R³ is

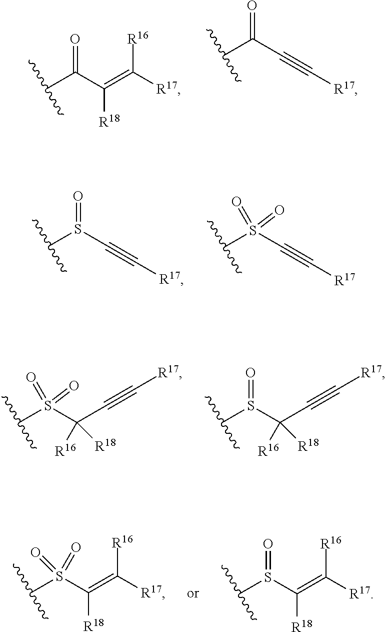

and
wherein R¹⁶, R¹⁷, and R¹⁸ are independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁴ is hydrogen, halogen or C1-C3 alkyl;
   each R⁵ is independently hydrogen or C1-C3 alkyl;
   each R⁸ is independently hydrogen, hydroxy, C1-C4 hydroxyalkyl or heteroaryl;
   each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, aryl, —CH₂—S(O)₂NH₂, or heteroaryl optionally independently substituted with 1-2 C1-C3 alkyl, —CN or C(O)NH₂,
   two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),
   two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
   two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;
   each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;
   each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
   R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;
   Q is N, C or 0, wherein if Q is C the 6-membered ring that includes Q is aromatic, and wherein if Q is 0 the 6-membered ring that includes Q is an oxane;
   each n is 0-3;
   is 1-6; and
   p is 1-8.

In certain embodiments, Y¹ and Y² join to form:

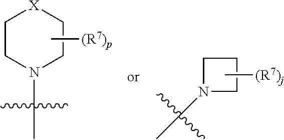

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—;
or Y¹ and Y² join to form:

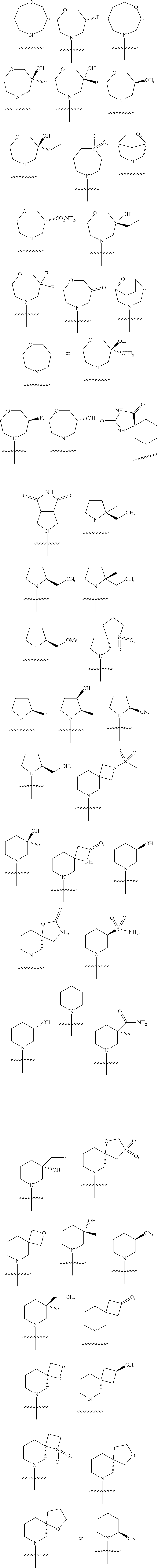

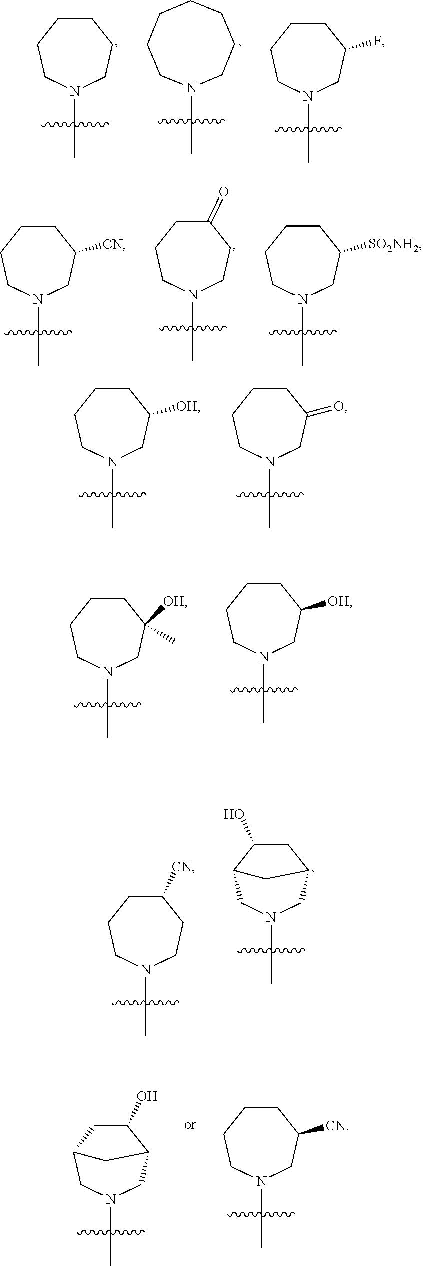

In certain embodiments, R³ is

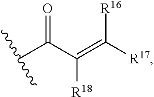

In certain embodiments, R¹⁶ is hydrogen, and R¹⁸ is hydrogen,

In certain embodiments, R¹⁶ is hydrogen, R¹⁷ is hydrogen, and R¹⁸ is unsubstituted methyl,

In certain embodiments, R¹⁶ is hydrogen, R¹⁷ is hydrogen, and R¹⁸ is —CN.

In certain embodiments, A is aryl, optionally substituted with 1-4 R¹; and Y¹ and Y² join to form:

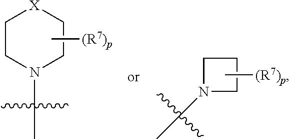

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—.

In certain embodiments, A is heteroaryl, optionally substituted with 1-4 R¹; and Y¹ and Y² join to form:

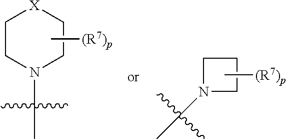

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—.

In certain embodiments, B is:

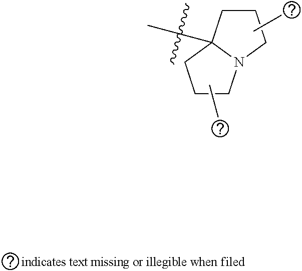

In certain embodiments, one or more of the following:
wherein A is naphthyl, indazolyl, benzothiophenyl, C1-C4 alkyl;
wherein at least one R² is halogen;
wherein at least one of R² and R³ is independently selected from the group consisting of ═CH₂, ═CHF, ═CF₂;
wherein R⁴ is halogen;
wherein one or both R⁶ are C1-C4 alkyl, hydrogen;
wherein two R⁷ on the same atom join to form a spirocyclic ring selected from C3-C6 cycloalkyl and heterocycle, where said spirocyclic ring is optionally substituted with one or more substituents selected from oxo (═O), halogen, hydroxy, C1-C3 alkyl and —O—(C1-C3 alkyl);
wherein two R⁷ on adjacent atoms join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R⁸;
wherein two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;
wherein at least one R⁸ is C1-C4 alkyl,
wherein at least one R⁸ is hydroxy or C1-C3 alkyl-hydroxy,
wherein one or two R⁸ are oxo (═O), and
wherein Y¹ and Y² join to form piperidine, azepane, azocane, thiazepine, diazepane, oxazepane, azetidine, pyrrolidine, piperazine bound to a fused ring via nitrogen or thiomorpholine.

In certain embodiments, the compound comprises the following formula:

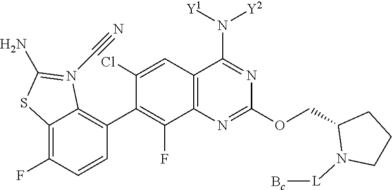

wherein Y¹ and Y² join to form a monocyclic or bicyclic substituted or unsubstituted heterocyclyl.

In certain embodiments, the compound comprises the following formula:

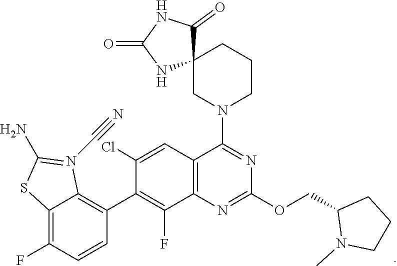

In certain embodiments, L-B_{C} is selected from the group consisting of:

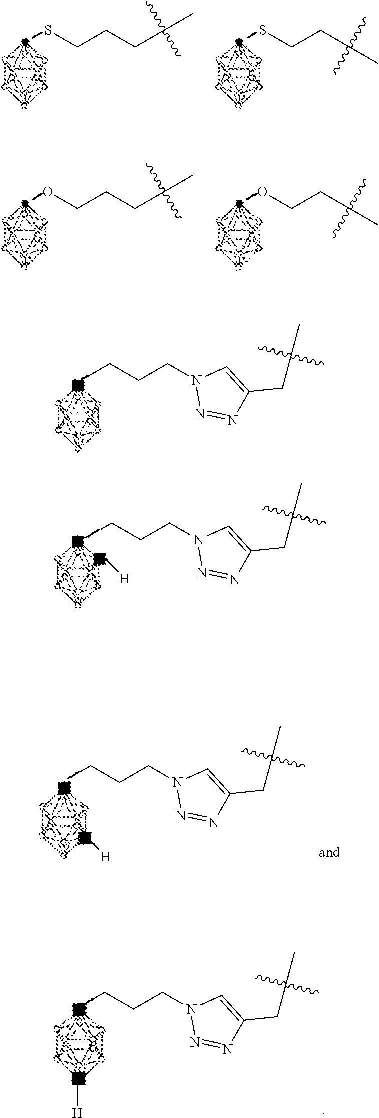

Unless otherwise stated, in certain embodiments, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, in certain embodiments, single stereochemical isomers as well as enantiomeric, diastereomeric, and geometric (or conformational) mixtures of the present compounds are within the scope of the invention. Unless otherwise stated, in certain embodiments, all tautomeric forms of the compounds of the invention are within the scope of the invention. Additionally, unless otherwise stated, in certain embodiments, structures depicted herein are also meant to include compounds that differ only in the presence of one or more isotopically enriched atoms. For example, in certain embodiments, compounds having the present structures including the replacement of hydrogen by deuterium or tritium, or the replacement of a carbon by a ¹³C- or ¹⁴C-enriched carbon and the replacement of boron by a ¹⁰B are within the scope of this invention. Such compounds are useful, for example, as analytical tools, as probes in biological assays, or as therapeutic agents in accordance with the present invention. In certain embodiments, a provided compound may be substituted with one or more deuterium atoms.

A compound of the present invention may be further tethered to a detectable moiety. It will be appreciated that such compounds are useful as imaging agents. One of ordinary skill in the art will recognize that a detectable moiety may be attached to a provided compound via a suitable substituent. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 200241:2596-99 and Sun et al., Bioconjugate Chem. 200617:52-57. As used herein, the term “detectable moiety” is used interchangeably with the term “label” and relates to any moiety capable of being detected, e.g., primary labels and secondary labels. Primary labels, such as radioisotopes (e.g., tritium, 32P, 33P, 35S, or 14C), mass-tags, and fluorescent labels are signal generating reporter groups which can be detected without further modifications. Detectable moieties also include luminescent and phosphorescent groups.

### B. Formulations and Pharmaceutically Acceptable Compositions

According to another embodiment, the invention provides a composition (e.g., a pharmaceutical composition) comprising a compound of this invention or a pharmaceutically acceptable derivative thereof and a pharmaceutically acceptable carrier, adjuvant, or vehicle.

The amount of compound in compositions of this invention is such that is effective to measurably bind a target protein, in a biological sample or in a patient. In certain embodiments, the amount of compound in compositions of this invention is such that is effective to measurably bind a protein, in a biological sample or in a patient. In certain embodiments, a composition of this invention is formulated for administration to a patient in need of such composition. In some embodiments, a composition of this invention is formulated for oral administration to a patient.

Compositions of the present invention may be administered orally, parenterally, by inhalation spray, topically, rectally, nasally, buccally, vaginally or via an implanted reservoir. In certain embodiments, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In certain embodiments, pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, in certain embodiments, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In certain embodiments, pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. In particular embodiments, topical application for the lower intestinal tract can be effected in a rectal suppository formulation (see above) or in a suitable enema formulation. Topically-transdermal patches may also be used. For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In another embodiment of the present invention, pharmaceutically acceptable compositions are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### III. Methods of the Present Invention

The compounds and compositions of the present invention target intracellular pathway proteins and may be administered to a subject/patient for modification with bi-modal boron neutron capture therapy functionality. Subsequent to administration, the patient may be irradiated with neutrons.

These pathway interacting borylated small molecules (“PIBS”) provide powerful dual acting therapeutic compounds for the treatment of cancer(s), immunological disorders, and other diseases, i.e., bi-modally treat cancer(s), immunological disorders, and other diseases by targeting intracellular disease pathways for modification, and which also serve to capture neutrons for boron neutron capture therapy.

In certain embodiments of the present invention, the intracellular protein participates in a cancer regulating pathway. In certain embodiments, the cancer regulating pathway may include, but is not limited to one or more of the following known signaling pathways that are known to participate in cancer regulation:
i) RAS/MAPK pathway
ii) PI3K/AKT/mTOR pathway
iii) Notch pathway
iv) Wnt/β-catenin pathway
v) P53 pathway
vi) TGF-β pathway
vii) Hippo YAP/TAZ pathway

In particular embodiments, the IPPM binds preferentially to an intracellular protein or molecule (e.g., gene encoding the protein) but may also bind to an extracellular protein or molecule (e.g., gene encoding the protein).

As such, one embodiment of the present invention provides a method of treating an intracellular protein mediated disorder, disease, or condition in a patient comprising administering to said patient a compound (e.g., a pharmaceutical composition) of the present invention, e.g., a therapeutically-effective amount of the compound. In certain embodiments, the method further comprises the step of irradiating the patient with neutrons, e.g., therapeutically (e.g., using a linear accelerator such as a cyclotron or linac that generates neutrons to interact with ¹⁰B to produce alpha particles that kill the cancer cells).

In certain embodiments of the present invention, the intracellular protein mediated disorder is associated with the signaling pathway selected from the group consisting of the RAS/MAPK pathway, the PI3K/AKT/mTOR pathway, the Notch pathway, the Wnt/3-catenin pathway, the P53 pathway, the TGF-β pathway, and the Hippo YAP/TAZ pathway. In certain embodiments, the intracellular protein mediated disorder is associated with the signaling pathway selected from the group consisting of the RAS/MAPK pathway, the PI3K/AKT/mTOR pathway, and the P53 pathway.

Another embodiment of the present invention provides a method of inducing apoptosis in a cell, the method comprising contacting the cell with a therapeutically-effective amount of a compound of the invention. In certain embodiments, the method further comprises the step of irradiating the cell with neutrons, e.g., therapeutically (e.g., using a linear accelerator such as a cyclotron or linac that generates neutrons to interact with ¹⁰B to produce alpha particles that kill the cancer cells).

In certain embodiments of the present invention, the disorder treated by the compounds and compositions of the present invention is a cancer, e.g., targeting and killing tumor cells. In certain embodiments, the disorder may include a cancer selected from the group consisting of a solid cancer, hematological cancer, lung cancer, pancreatic cancer, colorectal cancer, ovarian cancer, a cancer which is a metastatic cancer, or a cancer which is relapsed, a cancer which is refractory, a squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

### IV. Combination Therapies of the Present Invention

In certain embodiments, a provided combination, or composition thereof, is administered in combination with another therapeutic agent.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

### EXEMPLIFICATION

### General Synthetic Methods

Having thus described the invention in general terms, reference will now be made to exemplary embodiments, which are not intended to be limiting in any way. In this respect, the invention is capable of other embodiments and of being practiced and carried out in various ways. Also, it is to be understood that the phraseology and terminology employed herein are for the purpose of description and should not be regarded as limiting.

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A)₂ mM Ammonium Acetate in 0.1% Formic Acid in Water and (B)_{0.1}% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C182.1×30 mm columns packed with 5 mm C18-coated silica, eluting with 0.05 vol % NH3·H2O in water (solvent A) and acetonitrile (solvent B).

Prep HPLC Method: SepaFlash® Spherical C18, 20-45 μm, 100 Å; mobile phase: A for H₂O+0.1% NH₃·H₂O/H₂O and B for acetonitrile; gradient: B 5-95% in 20 min, flow rate: 80 mL/min; RT, wavelength: 220 nm/254 nm),
NMR Method: The 1H NMR spectra were recorded on a Bruker AV NEO or AV III 400 MHz/5 mm Probe. The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. The compounds and intermediates are numbered/designated within each Example, which numbering resets for each Example; wherein reference to each compound/intermediate would be referenced under each Example.

### Example I

### Synthesis of Compound 1

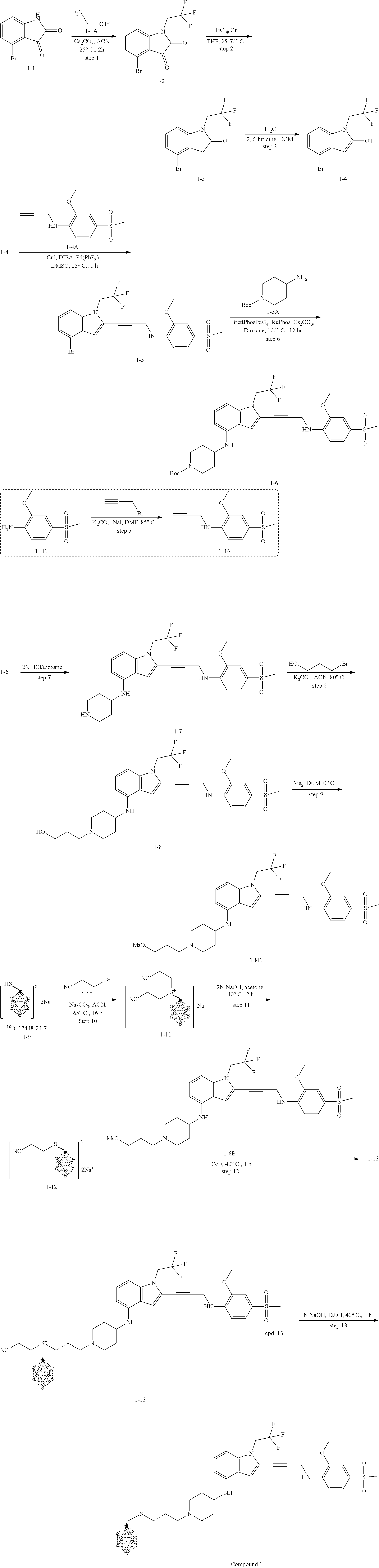

### Step 1: Synthesis of Intermediate 1-2

To a solution of Intermediate 1-1 (10 g, 44.24 mmol, 1 eq) in DMF (100 mL) was added Cs₂Co₃ (28.83 g, 88.48 mmol, 2 eq) and Intermediate 1-1A (12.32 g, 53.09 mmol, 1.1 eq). The mixture was stirred at 25° C. for 2 hrs. The reaction mixture was filtered. The filtrate was concentrated under reduced pressure to remove most of the DMF, then to the residue was added EA (200 mL) and the resulting mixture was stirred at 25° C. for 4 hrs. The mixture was filtered and the filtrate was washed with aqueous NaCl (100 mL*2), dried over Na₂SO₄, filtered and concentrated under reduced pressure to give a residue. To the residue was added co-solvent (PE/EA=50/1, 80 mL) and the mixture was stirred at 25° C. for 12 hrs. Then the mixture was filtered and the filter cake was concentrated under reduced pressure to afford Intermediate 1-2 (10 g, 73% yield) as a yellow solid. TLC Data: PE:EA=5:1, Rf=0.4

### Step 2: Synthesis of Intermediate 1-3

To a solution of Intermediate 1-2 (10 g, 32.46 mmol, 1 eq) in THF (300 mL) was added TiCl₄ (18.47 g, 97.39 mmol, 3 eq) and Zn powder (12.73 g, 194.77 mmol, 6 eq) at 25° C. The resulting mixture was stirred at 70° C. for 2 hrs. The reaction mixture was poured into HCl (1 M, 1 L) slowly at 25° C. and extracted with EA (300 mL*3), dried over anhydrous sodium sulfate, filtered and concentrated under reduced pressure to give a residue. The residue was purified by flash silica gel chromatography (ISCO®; 300 g SepaFlash® Silica Flash Column, Eluent of 0-50% Ethylacetate/Petroleum ethergradient @200 mL/min) to afford Intermediate 1-3 (8 g, 83.7% yield) as a yellow solid. TLC Data: PE:EA=3:1, Rf=0.4

### Step 3: Synthesis of Intermediate 1-4

To a solution of Intermediate 1-3 (8 g, 27.20 mmol, 1 eq) in DCM (80 mL) was added 2,6-dimethylpyridine (3.5 g, 32.65 mmol, 1.2 eq) and then Tf₂O (8.44 g, 29.92 mmol, 1.1 eq) was added at 0° C. The mixture was stirred at 25° C. for 1 hr. The reaction mixture was diluted with water (100 mL) and extracted with DCM (100 mL*3). The combined organic layers were dried over Na₂SO₄, filtered and concentrated under reduced pressure to give a residue. The residue was purified by flash silica gel chromatography (ISCO®; 220 g SepaFlash® Silica Flash Column, Eluent of 10~20% Ethylacetate/Petroleum ethergradient @200 mL/min) to afford Intermediate 1-4 (6 g, 51.7% yield) as a yellow solid. TLC Data: PE:EA=3:1, Rf=0.45

### Step 4: Synthesis of Intermediate 1-5

To a solution of Intermediate 1-4A (1 g, 4.18 mmol, 1 eq) in DMSO (10 mL) was added Intermediate 1-4 (1.96 g, 4.60 mmol, 1.1 eq), CuI (79.59 mg, 417.90 μmol, 0.1 eq), N-isopropylpropan-2-amine; tetrazole (2.15 g, 12.54 mmol, 3 eq) and Pd(PPh₃)₄ (48.29 mg, 41.79 μmol, 0.01 eq), the mixture is purged with N₂ for 3 times and then the resulting mixture was stirred at 25° C. for 1 hr under N₂. The reaction mixture was diluted with water (20 mL) and extracted with ethyl acetate (20 mL*3). The combined organic phases was washed with brine (10 mL*3), dried with anhydrous Na₂SO₄, filtered and concentrated in vacuum to get the residue. The residue was triturated with co-solvent (PE/EA=5/1, 100 mL) at 25° C. for 30 min and then filtered. The filter cake was concentrated under reduced pressure to afford Intermediate 1-5 (2.4 g, 4.10 mmol, 98.16% yield, 88.08% purity) as yellow oil.

Mass Data: LCMS: Retention time: 0.584 min, [M+H]⁺=829.9, 5-95AB_1 min_220&254.Icm; LCMS: Retention time: 0.586 min, [M+H]⁺=829.9, 5-95AB_1 min_220&254.Icm

NMR Data: ¹H NMR (400 MHz, DMSO-d6) δ=7.65-7.59 (m, 1H), 7.42-7.38 (m, 1H), 7.35 (d, J=7.5 Hz, 1H), 7.26 (d, J=1.7 Hz, 1H), 7.23-7.18 (m, 1H), 6.91 (d, J=8.4 Hz, 1H), 6.76 (s, 1H), 6.56-6.48 (m, 1H), 5.17-5.06 (m, 2H), 4.39 (br d, J=6.1 Hz, 2H), 3.89 (s, 3H), 3.09 (s, 3H)

### Step 5: Synthesis of Intermediate 1-4A

To a solution of Intermediate 1-48 (1.8 g, 8.94 mmol, 1 eq) in DMF (18 mL) was added 3-bromoprop-1-yne (3.19 g, 26.83 mmol, 2.31 mL, 3 eq) K₂CO₃ (3.71 g, 26.83 mmol, 3 eq) and NaI (2.68 g, 17.89 mmol, 2 eq). The mixture was stirred at 90° C. for 3 hr. LCMS (EW57260-3-P1A2) showed desired MW. TLC (PE:EA=1:1, Rf=0.5) showed a new spot. The reaction mixture was diluted with H₂O 10 mL and extracted with EA (10 mL*3). The combined organic layers were washed with sat.NaCl (5 mL*3), dried over Na₂SO₄, filtered and concentrated under reduced pressure to give a residue. The residue was purified by flash silica gel chromatography (ISCO®; 120 g SepaFlash® Silica Flash Column, Eluent of 0-80% Ethylacetate/Petroleum ethergradient 80 mL/min) and concentrated under vacuum to afford Intermediate 1-4A (1.1 g, 4.60 mmol, 51.39% yield) as yellow solid.

Mass Data: LCMS: Retention time: 0.425 min, [M+H]⁺=240.0, 5-95AB_1 min_220&254.Icm; LCMS: Retention time: 0.463 min, [M+H]⁺=240.1, 5-95AB_1 min_220&254.Icm

NMR Data: ¹H NMR (400 MHz, DMSO-d6) δ=7.44-7.34 (m, 1H), 7.23 (d, J=1.6 Hz, 1H), 6.75 (d, J=8.4 Hz, 1H), 6.37-6.25 (m, 1H), 4.04-3.97 (m, 2H), 3.88 (s, 3H), 3.15-3.03 (m, 4H)

### Step 6: Synthesis of Intermediate 1-6

To a solution of Intermediate 1-5 (2.4 g, 4.66 mmol, 1 eq) in dioxane (20 mL) was added Intermediate 1-5A (1.03 g, 5.12 mmol, 1.1 eq) dicyclohexyl-[3,6-dimethoxy-2-(2,4,6-triisopropylphenyl)phenyl]phosphane; methanesulfonate; [2-[2-(methylamino)phenyl]phenyl]palladium(1+) (214.35 mg, 232.85 μmol, 0.05 eq) and dicyclohexyl-[2-(2,6-diisopropoxyphenyl)phenyl]phosphane (217.32 mg, 465.71 μmol, 0.1 eq) Cs₂CO₃ (1.52 g, 4.66 mmol, 1 eq) the mixture was degassed and purged with N₂ for 3 times and then the mixture was stirred at 100° C. for 12 hr. The reaction mixture was quenched by adding water (10 mL) and extracted with EA (10 mL*3). The combined organic layers were washed with aqueous NaCl (10 mL), dried over Na₂SO₄, filtered and concentrated under reduced pressure to give a residue. The residue was purified by flash silica gel chromatography (ISCO®; 40 g SepaFlash® Silica Flash Column, Eluent of 0-60% Ethyl acetate/Petroleum ethergradient @80 mL/min) and concentrated under vacuum to afford Intermediate 1-6 (2.2 g, 3.47 mmol, 74.43% yield) as yellow solid.

Mass Data: LCMS: Retention time: 0.566 min, [M+H]⁺=635.2, 5-95AB_1 min_220&254.Icm; LCMS: Retention time: 0.562 min, [M+H]⁺=635.2, 5-95AB_1 min_220&254.Icm

NMR Data: ¹H NMR (400 MHz, DMSO-d6) δ=7.43-7.35 (m, 1H), 7.25 (d, J=1.9 Hz, 1H), 7.06 (s, 1H), 7.04-6.97 (m, 1H), 6.89 (d, J=8.4 Hz, 1H), 6.70 (d, J=8.1 Hz, 1H), 6.53-6.46 (m, 1H), 6.21 (d, J=7.9 Hz, 1H), 5.52 (d, J=8.1 Hz, 1H), 4.99-4.87 (m, 2H), 4.35 (d, J=6.3 Hz, 2H), 3.97-3.87 (m, 5H), 3.59-3.50 (m, 1H), 3.09 (s, 3H), 2.98-2.79 (m, 2H), 1.92 (br d, J=9.9 Hz, 2H), 1.40 (s, 9H), 1.37-1.26 (m, 2H)

### Step 7: Synthesis of Intermediate 1-7

To a solution of Intermediate 1-6 (500 mg, 787.76 μmol, 1 eq) in DCM (2 mL) was added HCl/dioxane (4 M, 2 mL, 10.16 eq). The mixture was stirred at 25° C. for 2 hr. LCMS (EW57260-20-P1A) showed a major peak of desired MW. The reaction mixture was concentrated under vacuum to afford Intermediate 1-7 (450 mg, crude, HCl) as a white solid.

Mass Data: LCMS: Retention time: 0.449 min, [M+H]⁺=535.1, 5-95AB_1 min_220&254.Icm

NMR Data: ¹H NMR (400 MHz, DMSO-d6) δ=9.25-8.86 (m, 2H), 7.46-7.39 (m, 1H), 7.33-7.19 (m, 4H), 6.94 (d, J=8.4 Hz, 1H), 6.88-6.79 (m, 1H), 5.16-5.05 (m, 2H), 3.64 (br d, J=2.5 Hz, 5H), 3.35 (br d, J=12.0 Hz, 3H), 3.18-3.12 (m, 3H), 3.08-2.89 (m, 3H), 2.12 (br d, J=11.3 Hz, 2H), 2.00-1.82 (m, 2H)

### Step 8: Synthesis of Intermediate 1-8

To a solution of Intermediate 1-7 (350 mg, 612.90 μmol, 1 eq, HCl) in ACN (4 mL) was added 3-bromopropan-1-ol (170.38 mg, 1.23 mmol, 110.85 μL, 2 eq) K₂CO₃ (423.53 mg, 3.06 mmol, 582.58 μL, 5 eq).The mixture was stirred at 80° C. for 1 hr. LCMS (EW57260-34-P1A1) showed a major peak of desired MW. The reaction mixture was added H₂O (2 mL) and used for purification. The residue was purified by reversed phase HPLC (120 g of SepaFlash® Spherical C18, 20-45 μm, 100 Å; mobile phase:A for H₂O+0.1% NH₃·H₂O/H₂O and B for acetonitrile; gradient: B 5-95% in 20 min, flow rate: 80 mL/min; column temperature: RT, wavelength: 220 nm/254 nm), concentrated under reduced pressure to remove MeCN and then extracted with DCM (10 mL*3) and washed with brine (8 mL). The organic layer was dried with anhydrous Na₂SO₄, filtered and concentrated under vacuum to afford Intermediate 1-8 (200 mg, 337.46 μmol, 55.06% yield) as yellow solid.

Mass Data: LCMS: Retention time: 0.445 min, [M+H]⁺=593.2, 5-95AB_1 min_220&254.Icm; LCMS: Retention time: 0.664 min, [M+H]+=593.4, 5-95CD_1.5 min_220&254_POS.Icm,

NMR Data: ¹H NMR (400 MHz, DMSO-d6) δ=7.39 (br d, J=8.4 Hz, 1H), 7.25 (s, 1H), 7.08 (s, 1H), 7.03-6.96 (m, 1H), 6.89 (br d, J=8.4 Hz, 1H), 6.67 (br d, J=8.3 Hz, 1H), 6.53-6.45 (m, 1H), 6.15 (br d, J=7.7 Hz, 1H), 5.48 (br d, J=7.3 Hz, 1H), 4.99-4.86 (m, 2H), 4.36 (br d, J=5.9 Hz, 2H), 3.89 (s, 3H), 3.47-3.42 (m, 4H), 3.09 (s, 3H), 2.86 (br d, J=10.4 Hz, 2H), 2.38-2.32 (m, 2H), 2.03-1.90 (m, 4H), 1.62-1.54 (m, 2H), 1.51-1.41 (m, 2H)

### Step 9: Synthesis of Intermediate 1-8B

To a solution of Intermediate 1-8 (180 mg, 303.71 μmol, 1 eq) in DCM (2 mL) was added methylsulfonyl methanesulfonate (63.49 mg, 364.45 μmol, 1.2 eq) and TEA (92.20 mg, 911.13 μmol, 126.82 μL, 3 eq). The mixture was stirred at 0° C. for 0.5 hr. LCMS (EW57260-35-P1A1) showed a major peak of desired MW. The reaction mixture was diluted with H₂O (2 mL), and extracted with DCM (10 mL*3). The combined organic layers was washed with sat.NaCl (3 mL*3), dried over Na₂SO₄, filtered and concentrated under reduced pressure to give Intermediate 1-8B (250 mg, crude) as yellow solid.

Mass Data: LCMS: Retention time: 0.459 min, [M+H]⁺=671.1, 5-95AB_1 min_220&254.Icm; LCMS: Retention time: 0.459 min, [M+H]⁺=671.1, 5-95AB_1 min_220&254.Icm

NMR Data: ¹H NMR (400 MHz, CHLOROFORM-d) δ=7.56-7.51 (m, 1H), 7.50-7.46 (m, 1H), 7.28 (br s, 1H), 7.26 (br s, 1H), 7.23 (d, J=1.7 Hz, 1H), 7.18-7.12 (m, 1H), 7.11-7.05 (m, 1H), 6.82 (d, J=8.3 Hz, 1H), 6.76 (d, J=8.4 Hz, 1H), 6.72 (s, 1H), 6.65 (br d, J=8.3 Hz, 1H), 6.59 (br d, J=8.3 Hz, 1H), 6.32-6.27 (m, 1H), 6.17 (d, J=7.8 Hz, 1H), 5.45 (br d, J=7.1 Hz, 1H), 5.29-5.24 (m, 1H), 5.19-5.13 (m, 1H), 4.65-4.52 (m, 4H), 4.38-4.25 (m, 7H), 4.09-4.01 (m, 2H), 3.94 (s, 3H), 3.85 (s, 3H), 3.84-3.74 (m, 3H), 3.63-3.54 (m, 1H), 3.51-3.43 (m, 1H), 3.29-3.20 (m, 2H), 3.03 (d, J=3.2 Hz, 6H), 2.79 (s, 3H), 2.77 (s, 3H), 2.67-2.62 (m, 1H), 2.52-2.45 (m, 3H), 2.42-2.30 (m, 8H), 2.23-2.09 (m, 6H), 2.01-1.92 (m, 5H), 1.59-1.48 (m, 2H)

### Step 10: Synthesis of Intermediate 1-11

To a solution of Intermediate 1-9 (100.00 mg, 475.87 μmol, 1 eq) in ACN (10 mL) was added Na₂CO₃ (53.46 mg, 504.43 μmol, 1.06 eq).The mixture was stirred at 65° C. for 16 hr. The reaction mixture was filtered and evaporated under reduced pressure to afford Intermediate 1-11 (300 mg, crude, Na+) as a colorless oil.

NMR Data: ¹H NMR (400 MHz, ACETONITRILE-d3) δ=3.43-3.15 (m, 4H), 3.03-2.94 (m, 4H), 1.69-0.87 (m, 11H)

### Step 11: Synthesis of Intermediate 1-12

To a solution of Intermediate 1-11 (300 mg, 1.02 mmol, 1 eq, Na+) in ACETONE (2 mL) was added NaOH (2 M, 1 mL, 1.96 eq).The mixture was stirred at 40° C. for 1 hr. The reaction mixture was adjusted PH=7 with 1 N HCl and evaporated under reduced pressure to get the residue, the residue was added EtOH(10 mL) and filtered, the filtrate was concentrated under reduced pressure to afford Intermediate 1-12 (250 mg, crude, 2Na+) as white solid.

NMR Data: ¹H NMR (400 MHz, ACETONITRILE-d3) δ=3.73-3.64 (m, 2H), 2.69-2.64 (m, 2H), 1.63-0.51 (m, 11H)

### Step 12: Synthesis of Intermediate 1-13

To a solution of Intermediate 1-12 (110.00 mg, 125.30 μmol, 1 eq, 2Na+) in DMF (1 mL) was added 3 Intermediate 1-8B (100.86 mg, 150.36 μmol, 1.2 eq) and the mixture was stirred at 40° C. for 1 hr. LCMS(EW57260-55-P1A) showed 71% of desired MW. The reaction mixture was purified directly and no work up. The residue was purified by reversed phase HPLC(80 g of SepaFlash® Spherical C18, 20-45 μm, 100 Å; mobile phase:A for H2O+0.1% NH₃·H₂O/H₂O and B for acetonitrile; gradient: B 5-95% in 20 min, flow rate: 80 mL/min; column temperature: RT, wavelength: 220 nm/254 nm), concentrated under reduced pressure to remove MeCN and then lyophilization to afford Intermediate 1-13 (50 mg, 59.59 μmol, 47.56% yield, 2Na+) as a yellow solid.

Mass Data: LCMS: Retention time=0.549 min,[M+H]+=792.9, 5-95CD_1.5 min_220&254_NEG.Icm.

NMR Data: ¹H NMR (400 MHz, ACETONITRILE-d3) δ=7.45 (br d, J=8.4 Hz, 1H), 7.27 (s, 1H), 7.17-7.07 (m, 1H), 6.91 (d, J=8.4 Hz, 1H), 6.84 (s, 1H), 6.74 (br d, J=8.0 Hz, 1H), 6.30 (d, J=7.8 Hz, 1H), 5.70-5.57 (m, 1H), 4.81-4.71 (m, 2H), 4.57 (br d, J=7.3 Hz, 1H), 4.36 (d, J=6.4 Hz, 2H), 3.92 (s, 3H), 3.76-3.67 (m, 1H), 3.53-3.43 (m, 2H), 3.40-3.33 (m, 1H), 3.24-3.05 (m, 6H), 2.99 (s, 6H), 1.84-1.73 (m, 2H), 1.67-0.86 (m, 15H).

### Step 13: Synthesis of Compound 1

To a solution of Intermediate 1-13 (50 mg, 59.59 μmol, 1 eq, 2Na+) in EtOH (1 mL) was added NaOH (1 M, 1 mL, 16.78 eq) and the mixture was stirred at 40° C. for 1 hr. LCMS(EW57260-56-P1A) showed a major peak of desired MW. The reaction mixture was purified directly and no work up. The residue was purified by reversed phase HPLC (80 g of SepaFlash® Spherical C18, 20-45 μm, 100 Å; mobile phase:A for H₂O+0.1% NH₃·H₂O/H₂O and B for acetonitrile; gradient: B 5-95% in 20 min, flow rate: 80 mL/min; column temperature: RT, wavelength: 220 nm/1254 nm), concentrated under reduced pressure to remove MeCN and then lyophilization to afford Compound 1 (20 mg, 27.06 μmol, 45.42% yield) as a yellow solid.

Mass Data: LCMS: Retention time=0.424 min,[M+H]+=739.7, 5-95CD_1.5 min_220&254_POS.Icm; LCMS: Retention time=0.427 min,[M+H]+=739.4, 5-95CDS1.5 min_220&254_POS_2000.Icm.

NMR Data: 1H NMR (400 MHz, ACETONITRILE-d3) δ=7.49-7.41 (in, 1H), 7.26 (d, J=1.8 Hz, 1H), 7.17-7.04 (in, 1H), 6.94-6.85 (m, 2H), 6.77-6.70 (in, 1H), 6.31 (in, 1H), 6.03-5.85 (m, 2H), 5.67-5.54 (in, 1H), 4.78-4.71 (m, 2H), 4.37 (m, 2H), 3.92 (s, 3H), 3.89 (br d, J=13.4 Hz, 1H), 3.74-3.57 (in, 1H), 3.31-3.25 (m, 2H), 2.99 (s, 3H), 2.97-2.92 (in, 1H), 2.80-2.74 (m, 2H), 2.37-2.22 (m, 2H), 2.04-1.97 (m, 2H), 1.90-1.82 (m, 2H), 1.71-1.45 (m, 2H), 1.41-0.76 (m, 1H).

### Example 2

### Synthesis of Compound 2a, 2a [O], 2b and 2b [O]

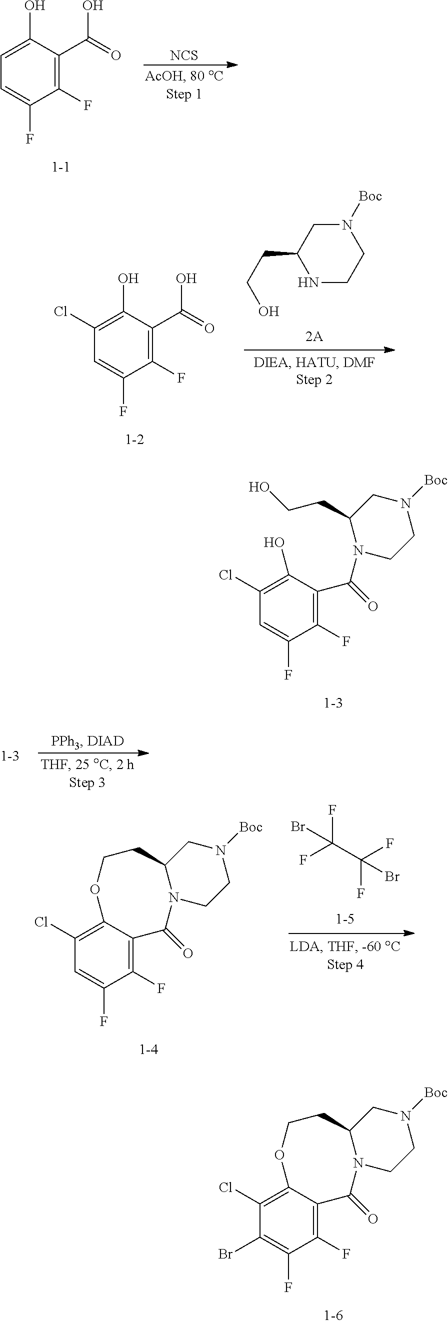

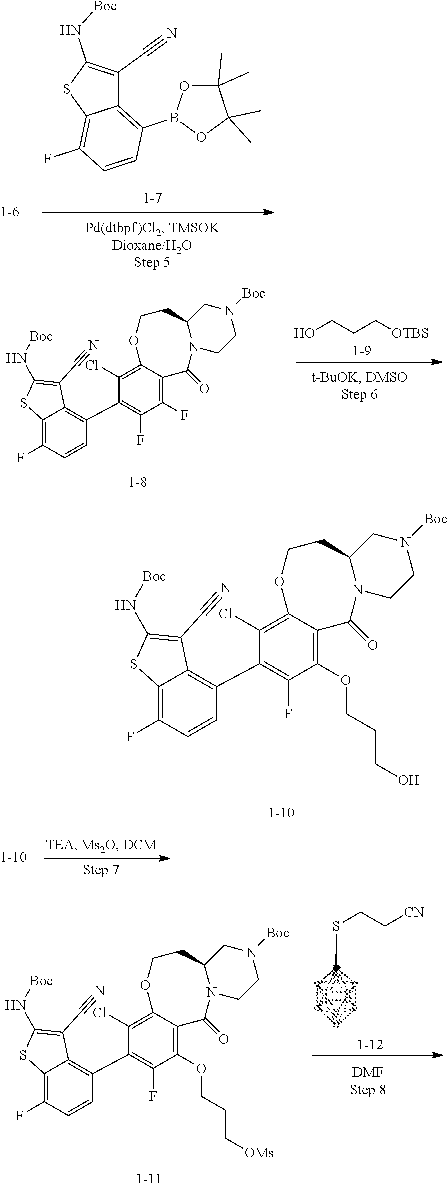

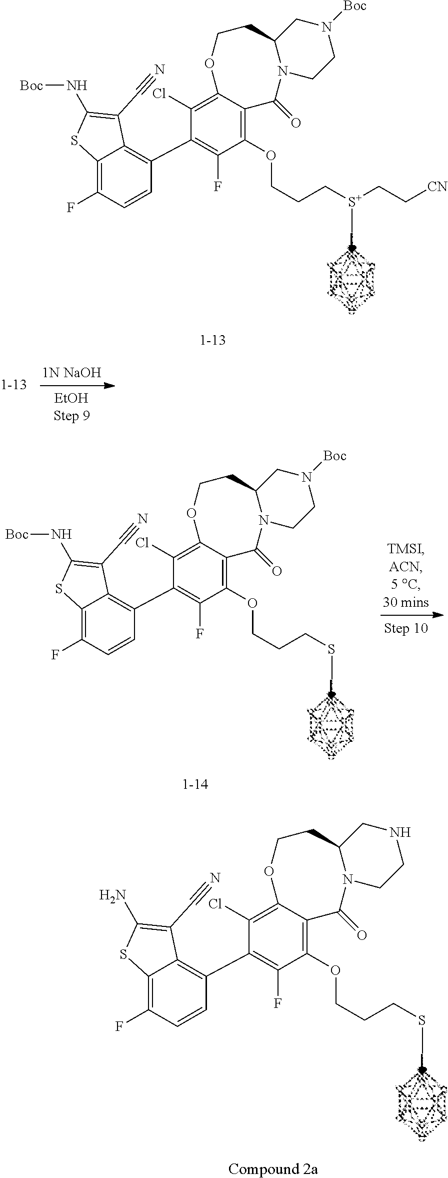 [SMILES: C[S+](CCC#N)CCCOc1c(F)c(-c2ccc(F)c3sc(NC(=O)OC(C)(C)C)c(C#N)c23)c(Cl)c2c1C(=O)N1CCN(C(=O)OC(C)(C)C)C[C@@H]1CCO2 ; "1N NaOH" ; CSCCCOc1c(F)c(-c2ccc(F)c3sc(NC(=O)OC(C)(C)C)c(C#N)c23)c(Cl)c2c1C(=O)N1CCN(C(=O)OC(C)(C)C)C[C@@H]1CCO2 ; CSCCCOc1c(F)c(-c2ccc(F)c3sc(N)c(C#N)c23)c(Cl)c2c1C(=O)N1CCNC[C@@H]1CCO2]

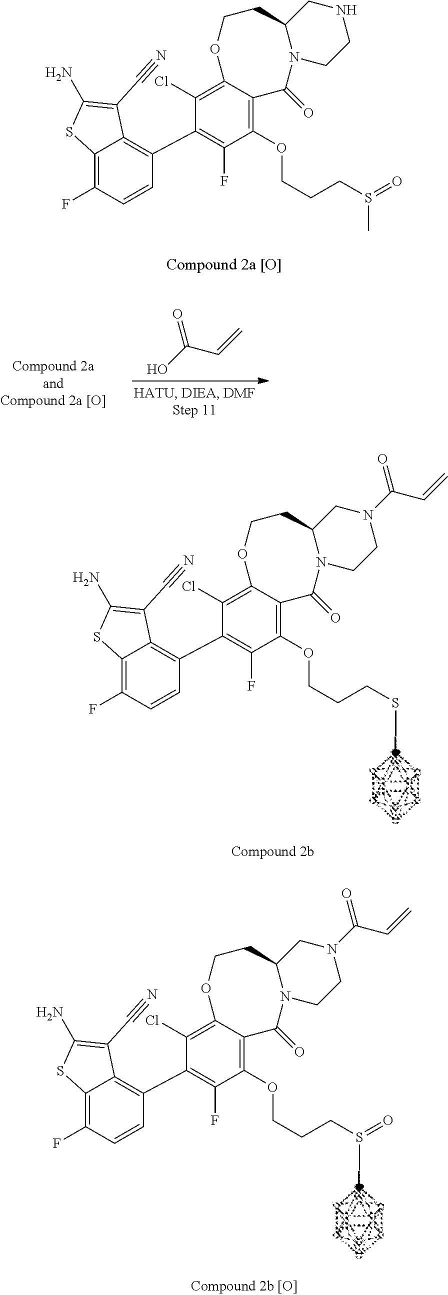

### Step 1: Synthesis of Intermediate 1-2

To a solution of Intermediate 1-1 (15 g, 86.16 mmol, 1 eq) in AcOH (75 mL) was added NCS (13.81 g, 103.39 mmol, 1.2 eq). The mixture was stirred at 60° C. for 3 hr. LCMS showed desired mass was detected. The reaction mixture was concentrated under vacuum to afford the crude product. The residue was purified by reversed phase column HPLC (0.1% FA condition). The eluent was combined and concentrated under vacuum to remove MeCN. The precipitate was collected by filter and then dried by oil pump to afford Intermediate 1-2 (11 g, 52.61 mmol, 61.07% yield, 99.75% purity) was obtained as a white solid.

Mass Found: LCMS: Rt: 0.198 min, [M−H]⁺=207.0, 0-60CD_0_1.5 min_220&254_NEG.Icm; LCMS: Rt: 0.174 min, [M+H]⁺=207.0, 0-60CD_0_1.5 min_220&254_NEG.Icm

NMR Data: ¹H NMR (400 MHz, DMSO-d6) δ=8.02-7.85 (m, 1H)

### Step 2: Synthesis of Intermediate 1-3

To a solution of Intermediate 1-2 (5 g, 23.98 mmol, 1 eq) in THF (80 mL) was added DIEA (15.49 g, 119.88 mmol, 20.88 mL, 5 eq) and HATU (13.67 g, 35.96 mmol, 1.5 eq). Then the mixture was stirred at 25° C. for 1 h. Then Intermediate 1-2a (6.63 g, 28.77 mmol, 1.2 eq) in THF (20 mL) was added to the above mixture and stirred for another 12 h at 25° C. LCMS showed desired mass was detected. The 2 batches were combined and the mixture was diluted with H₂O (400 mL) and extracted with EA (300 mL*2). The combined organic phase was dried over anhydrous Na₂SO₄, filtered and concentrated to afford the residue. The residue was purified by reversed phase column HPLC (0.1% FA condition). The eluent was combined and concentrated under vacuum to remove MeCN. The aqueous phase was extracted with EA (300 mL*2). The organic phase was dried over anhydrous Na₂SO₄, filtered and concentrated to afford Intermediate 1-3 (4.4 g, 8.89 mmol, 24.93% yield, 85% purity) was obtained as a white solid.

Mass Found: LCMS: Rt: 0.480 min, [M−55]⁺=365.0, 5-95AB_0.8 min_220&254.Icm; LCMS: Rt: 0.566 min, [M−55]⁺=365.1, 5-95AB_0.8 min_220&254.Icm

### Step 3: Synthesis of Intermediate 1-4

To a solution of PPh₃ (2.99 g, 11.41 mmol, 2 eq) in THF (30 mL) was added DIAD (2.31 g, 11.41 mmol, 2.21 mL, 2 eq) at 0° C. Then the mixture was stirred at 0° C. for 15 min. To the above mixture was added Intermediate 1-3 (2.4 g, 5.70 mmol, 1 eq) in THF (60 mL) at 0° C. dropwise over 1h. LCMS showed desired mass was detected. The reaction diluted with H₂O (100 mL) and extracted with EA (100 mL*2). The combined organic phase was dried over anhydrous Na₂SO₄, filtered and concentrated to afford the residue. The residue was purified by flash silica gel chromatography (ISCO®; 330 g SepaFlash® Silica Flash Column, Eluent of 0~60% Ethyl acetate/Petroleum ether gradient @100 mL/min). The eluent was combined and concentrated to afford Intermediate 1-4 (900 mg, 2.23 mmol, 39.10% yield, 99.80% purity) was obtained as a white solid.

Mass Found: LCMS: Rt: 0.821 min, [M−55]⁺=347.0, 5-95AB_1.5 min_220&254.Icm; LCMS: Rt: 0.993 min, [M−55]⁺=347.1, 5-95AB_1.5 min_220&254.Icm

¹H NMR (400 MHz, DMSO-d6) δ=7.95-7.86 (m, 1H), 4.33-4.17 (m, 2H), 4.16-4.08 (m, 1H), 3.98 (br d, J=2.3 Hz, 1H), 3.81-3.71 (m, 1H), 3.13-2.79 (m, 3H), 2.26-2.12 (m, 1H), 1.69 (br d, J=14.4 Hz, 1H), 1.40 (s, 9H), 1.26-1.19 (m, 1H)

### Step 4: Synthesis of Intermediate 1-6

To a solution of Intermediate 1-4 (1 g, 2.48 mmol, 1 eq) in THF (20 mL) was added LDA (2 M, 2.48 mL, 2 eq) at −60° C. Then the reaction mixture was stirred at -60° C. for 1 h. Then Intermediate 1-5 (774.01 mg, 2.98 mmol, 1.2 eq) was added to the reaction mixture and warmed to 0° C. Then the reaction mixture was stirred at 0° C. for 1h. LCMS showed desired mass and multiple peaks of by-product were detected. The mixture was quenched with saturated NH₄Cl aq. (50 mL) and extracted with EA (50 mL*2). The combined organic phase was dried over anhydrous Na₂SO₄, filtered and concentrated to afford the residue. The residue was purified by reversed phase column HPLC (0.1% FA condition). The eluent was combined and concentrated to remove MeCN. Then the aqueous phase was extracted with EA (10 mL*2). The combined organic phase was dried over anhydrous Na₂SO₄, filtered and concentrated to afford Intermediate 1-6 (0.5 g, 912.36 μmol, 36.75% yield, 87.9% purity) was obtained as a yellow solid.

Mass Found: LCMS: Rt: 0.611 min, [M−55]⁺=427.0, 5-95AB_1 min_220&254.Icm; LCMS: Rt: 0.612 min, [M−55]⁺=427.0, 5-95AB_1 min_220&254.Icm

### Step 5: Synthesis of Intermediate 1-8

To a solution of Intermediate 1-6 (0.1 g, 207.59 μmol, 1 eq) and Intermediate 1-7 (260.50 mg, 622.77 μmol, 3 eq) in dioxane (2 mL) and H2O (0.4 mL) was added Pd(dtbpf)Cl₂ (27.06 mg, 41.52 μmol, 0.2 eq) and TMSOK (79.89 mg, 622.77 μmol, 3 eq). Then the reaction was degassed by N₂ three times and then stirred at 100° C. for 2 hr. LCMS showed desired mass was detected. The reaction mixture was diluted with H₂O (50 mL), then extracted with EA (50 mL*3). The combined organic phase was dried over anhydrous Na₂SO₄, filtered and concentrated to afford the residue. The residue was purified by reversed phase column HPLC (0.1% FA condition). The eluent was combined and concentrated to remove MeCN. The aqueous phase was extracted with EA (10 mL*2). The combined organic phase was dried over anhydrous Na₂SO₄, filtered and concentrated to afford Intermediate 1-8 (180 mg, 254.50 μmol, 40.87% yield, 98% purity) was obtained as a yellow solid.

Mass Found: LCMS: Rt: 1.168 min, [M+H]⁺=693.2, 5-95AB_1.5 min_220&254.Icm; LCMS: Rt: 0.675/0.685 min, [M+H]⁺=693.2, 5-95AB_1 min_220&254.Icm

### Step 6: Synthesis of Intermediate 1-10

To a solution of Intermediate 1-9 (219.70 mg, 1.15 mmol, 5 eq) in DMSO (3 mL) was added t-BuOK (129.51 mg, 1.15 mmol, 5 eq) and Intermediate 1-8 (0.16 g, 230.84 μmol, 1 eq). Then the mixture was stirred at 60° C. for 2h. LCMS showed desired mass was detected. The reaction was diluted with H₂O (10 mL) and extracted with EA (10 mL*2). The combined organic phase was dried over anhydrous Na₂SO₄, filtered and concentrated to afford the residue. The residue was purified by flash silica gel chromatography (ISCO®; 40 g SepaFlash® Silica Flash Column, Eluent of 0~100% Ethyl acetate/Petroleum ether gradient @80 mL/min). The eluent was combined and concentrated to afford Intermediate 1-10 (130 mg, 166.57 μmol, 72.16% yield, 96% purity) was obtained as a white solid.

Mass Found: LCMS: Rt: 0.647 min, [M+H]⁺=749.3, 5-95AB_1 min_220&254.Icm LCMS: Rt: 0.633 min, [M+H]⁺=749.2, 5-95AB_1 min_220&254.Icm

### Step 7: Synthesis of Intermediate 1-11

To a solution of Intermediate 1-10 (170 mg, 226.90 μmol, 1 eq) in DCM (2 mL) was added TEA (114.80 mg, 1.13 mmol, 157.91 μL, 5 eq) and Ms₂O (118.58 mg, 680.71 μmol, 3 eq) at 0° C. Then the reaction was stirred at 0° C. for 0.5 hr. LCMS showed desired mass was detected. The reaction was diluted with H₂O (5 mL) and extracted with DCM (5 mL*2). The combined organic phase was dried over anhydrous Na₂SO₄, filtered and concentrated to afford the crude product Intermediate 1-11 (210 mg, crude) as a yellow solid.

Mass Found: LCMS: Rt: 0.705 min, [M+H]⁺=827.1, 5-95AB_1 min_220&254.Icm

### Step 8: Synthesis of Intermediate 1-13

To a solution of Intermediate 1-11 (0.17 g, 205.49 μmol, 1 eq) in DMF (2 mL) was added Intermediate 1-12 (270 mg, 618.19 μmol, 3.01 eq). Then the reaction was heated to 60° C. for 8 hr. LCMS showed desired mass was detected. The reaction was purified by reversed phase column directly. The residue was purified by reversed phase column HPLC (0.1% NH3·H2O condition). The eluent was combined and concentrated to remove MeCN, then lyophilized to afford the Intermediate 1-13 (42 mg, 44.18 μmol, 21.50% yield) was obtained as a yellow solid.

Mass Found: LCMS: Rt: 0.491 min, [M−H]⁺=948.4, 5-95CD_1.5 min_220&254_NEG.Icm; LCMS: Rt: 0.575 min, [M−H]⁺=948.4, 5-95CD_1.5 min_220&254_NEG.Icm

### Step 9: Synthesis of Intermediate 1-14

To a solution of Intermediate 1-13 (40 mg, 42.08 μmol, 1 eq) in EtOH (0.5 mL) was added NaOH (1 M, 0.5 mL, 11.88 eq). Then the reaction mixture was stirred at 40° C. for 1 hr. LCMS showed major peak with desired mass was detected. The reaction mixture was purified by reversed phase column HPLC (0.1% NH₃·H₂O condition). The eluent was combined and dried by lyophilization to afford Intermediate 1-14 (25 mg, 27.56 μmol, 65.49% yield, 98.7% purity) was obtained as a white solid.

Mass Found: LCMS: Rt: 0.439 min, [M−H]⁺=895.3, 5-95CD_1.5 min_220&254_NEG.Icm; LCMS: Rt: 0.402 min, [M−H]⁺=895.3, 5-95CD_1.5 min_220&254_NEG.Icm

### Step 10: Synthesis of Compound 2a Batch-1 and Compound 2a 101

To a solution of Intermediate 1-14 (20.00 mg, 22.34 μmol, 1 eq) in MeCN (1 mL) was added TMSI (67.04 mg, 335.03 μmol, 45.60 μL, 15 eq) at 0° C. Then the reaction was stirred for 0.5 hr at 0° C. LCMS showed desired mass was detected. The reaction mixture was purified was purified by reversed phase column HPLC (0.1% NH₃·H₂O condition, 0-30% MeCN). The eluent was combined and dried by lyophilization to afford: Compound 2a_Batch-1 (3.28 mg, 4.32 μmol, 19.33% yield, 91.66% purity) was obtained as a white solid.

Compound 2a [0](3.94 mg, 5.44 μmol, 24.37% yield, 98.24% purity) was obtained as a white solid.

Mass Found: LCMS: Rt: 0.309 min, [M−H]⁺=695.3, Rt: 0.637 min, [M−H]⁺=710.2, 5-95CD_1.5 min_220&254_NEG.Icm; Special LCMS: Rt: 2.111/2.178 min, [M−H]⁺=693.6, HSS_T3_50_4.6 mm_3.5 μm_5-95CD_4.5minU_M1_1.2_E_NEG.amx; Special LCMS: Rt: 2.004/2.032/2.068 min, [M−H]⁺=710.6, XBridge_C18_50_2.1 mm_5 μm_5-95CD_4.5minU_0.8_E_NEG.amx

NMR data: ¹H NMR (400 MHz, DMSO-d6) δ=8.27-7.97 (m, 2H), 7.35-7.26 (m, 1H), 7.15-7.06 (m, 1H), 4.54-4.35 (m, 2H), 4.33-3.92 (m, 4H), 3.15-3.00 (m, 4H), 2.31-2.22 (m, 2H), 1.93-1.74 (m, 2H), 1.69-1.56 (m, 1H), 1.43-0.46 (m, 14H)

¹H NMR (400 MHz, DMSO-d6) δ=8.13 (br d, J=16.0 Hz, 2H), 7.35-7.18 (m, 1H), 7.17-7.09 (m, 1H), 4.51-4.32 (m, 2H), 4.24-4.03 (m, 4H), 3.11-2.96 (m, 3H), 2.94-2.78 (m, 4H), 2.18-1.98 (m, 2H), 1.72-0.75 (m, 14H)

### Step 10: Synthesis of Compound 2a Batch-2 and Compound 2a [O]

To a solution of Intermediate 1-14 (24 mg, 26.80 μmol, 1 eq) in MeCN (1 mL) was added TMSI (80.44 mg, 402.04 μmol, 54.72 μL, 15 eq) at 0° C. Then the reaction was stirred for 0.5 hr at 0° C. LCMS showed desired mass was detected. The reaction mixture was purified by reversed phase column HPLC (0.1% NH₃·H₂O condition, 0-30% MeCN). The eluent was combined and dried by lyophilization to afford the products. The combined solid was re-purified by reversed phase column HPLC (0.1% NH₃·H₂O condition) twice. The eluent was combined and dried by lyophilization to afford the product which was detected by LCMS. After lyophilization, Compound 2a_Batch-2 (9 mg, 12.44 μmol, 57.65% yield, 96.09% purity) was obtained as a white solid.

Mass Found: LCMS: Rt: 0.348 min, [M−H]⁺=693.3, 5-95CD_1.5 min_220&254_NEG.Icm; LCMS: Rt: 0.358/0.379 min, [M−H]⁺=695.3, 5-95CD_1.5 min_220&254_NEG.Icm; Special LCMS: Rt: 2.153/2.22 min, [M−H]⁺=693.3, HSS_T3_50_4.6 mm_3.5 μm_5-95CD_4.5minU_M1_1.2_E_NEG.amx

NMR data: ¹H NMR (400 MHz, DMSO-d6) δ=8.28-7.99 (m, 2H), 7.37-7.27 (m, 1H), 7.15-7.08 (m, 1H), 4.67-4.21 (m, 3H), 4.18-3.91 (m, 3H), 3.05 (br s, 4H), 2.32-2.22 (m, 2H), 1.91-1.76 (m, 2H), 1.70-1.59 (m, 1H), 1.48-0.45 (m, 14H)

### Example 3

### Synthesis of Compound 3

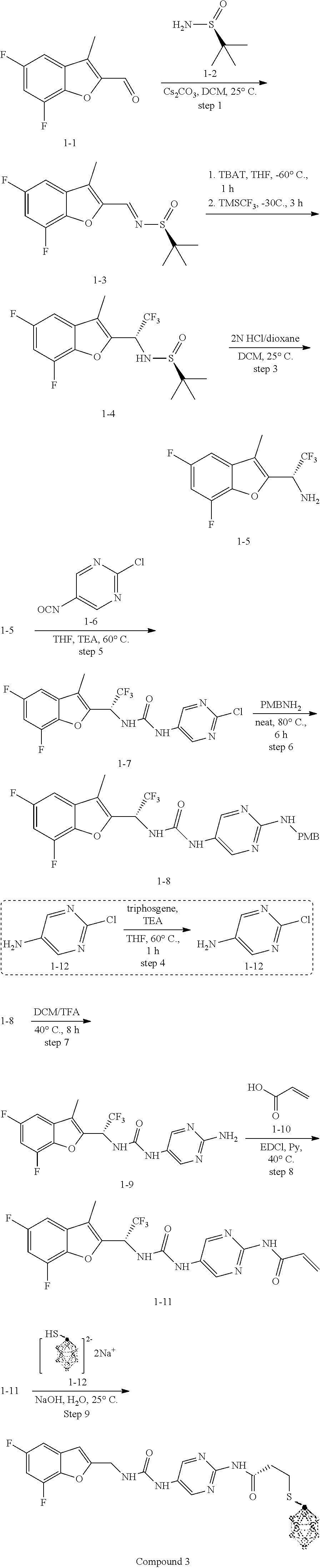

### Step 1: Synthesis of Intermediate 1-3

To a solution of Intermediate 1-1 (1 g, 5.10 mmol, 1 eq) in DCM (10 ml-) was added Cs₂Co₃ (1.99 g, 6.12 mmol, 1.2 eq). The mixture was stirred at 25° C. for 0.5 h. Then Intermediate 1-2 (679.69 mg, 5.61 mmol, 1.1 eq) was added. The mixture was stirred at 25° C. for 1.5 h. LCMS (EW57260-177-P1A) showed a major peak of desired MW. TLC (PE:EA=1:1, Rf=0.6) showed a major spot. The reaction mixture was diluted with H₂O 10 mL and extracted with DCM (10 mL*3). The combined organic layers were washed with sat.NaCl (10 mL*1), dried over Na₂SO₄, filtered and concentrated under reduced pressure to give a residue. The residue was purified by flash silica gel chromatography (ISCO®; 40 g SepaFlash® Silica Flash Column, Eluent of 0~50% Ethylacetate/Petroleum ether gradient @60 mL/min) and concentrated under vacuum to afford Intermediate 1-3 (1.5 g, 5.01 mmol, 98.29% yield) as yellow solid.

Mass Data: LCMS: Retention time: 0.571 min, [M+H]⁺=299.9, 5-95AB_1 min_220&254.Icm; LCMS: Retention time: 0.567 min, [M+H]⁺=300.0, 5-95AB_1 min_220&254.Icm

### Step 2: Synthesis of Intermediate 1-4

To a solution of Intermediate 1-3 (1.5 g, 5.01 mmol, 1 eq) in THF (12 mL) was added difluoro(triphenyl)silanuide; tetrabutylammonium (4.06 g, 7.52 mmol, 1.5 eq) at −60° C. and the reaction mixture stirred at −60° C. for 1 h, then TMSCF₃ (2.85 g, 20.04 mmol, 4 eq) in THF (3 mL) was added at −60° C. The mixture was stirred at −30° C. for 3 h. TLC (PE:EA=1:1, Rf=0.4) showed a new spot. The combined reaction mixture was diluted with water (10 mL) and was extracted with ethyl acetate (15 mL*3). The combined organic phases was washed with brine (8 mL*3), dried with anhydrous Na₂SO₄, filtered and concentrated in vacuum to get the residue. The residue was purified by flash silica gel chromatography (ISCO®; 40 g SepaFlash® Silica Flash Column, Eluent of 0~60% Ethylacetate/Petroleum ethergradient @60 mL/min) and concentrated under vacuum to afford Intermediate 1-4 (1.4 g, 3.25 mmol, 64.95% yield, 85.869% purity) as yellow oil.

Mass Data: LCMS: Retention time: 0.615 min, [M+H]⁺=370.0, 5-95AB_1 min_220&254.Icm

### Step 3: Synthesis of Intermediate 1-5

To a solution of Intermediate 1-4 (1.5 g, 4.06 mmol, 1 eq) in DCM (7 mL) was added HCl/dioxane (4 M, 7.50 mL, 7.39 eq). The mixture was stirred at 25° C. for 1 hr.

TLC (PE:EA=1:1, Rf=0.16) showed a new spot. The reaction mixture was quenched with MeOH (1 mL) and the pH was adjusted to 7-8 with NH₃·H₂O, and extracted with DCM 30 mL (10 mL*3). The combined organic layers was washed with sat.NaCl (5 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0~60% Ethyl acetate/Petroleum ethergradient @80 mL/min). and concentrated under vacuum to afford Intermediate 1-5 (900 mg, 3.09 mmol, 76.15% yield, 91.116% purity) as yellow oil.

Mass Data: LCMS: Retention time: 0.524 min, [M−17]⁺=248.9, 5-95AB_1 min_220&254.Icm

### Step 4: Synthesis of Intermediate 1-6

To a solution of Intermediate 1-12 (2 g, 15.44 mmol, 1 eq) in THF (200 mL) was added bis(trichloromethyl) carbonate (4.58 g, 15.44 mmol, 1 eq) and TEA (4.69 g, 46.32 mmol, 6.45 mL, 3 eq). The mixture was stirred at 60° C. for 1 hr. TLC (PE:EA=3:1) showed Intermediate 1-12 (2 g, 15.44 mmol, 1 eq) was consumed. The reaction mixture was concentrated under vacuum to afford Intermediate 1-6 (10 g, crude). The crude product was used for next step directly.

### Step 5: Synthesis of Intermediate 1-7

To a solution of Intermediate 1-5 (900 mg, 3.39 mmol, 1 eq) in THF (27 mL) was added TEA (1.03 g, 10.18 mmol, 1.42 mL, 3 eq) and Intermediate 1-6 (8.45 g, 54.30 mmol, 16 eq). The mixture was stirred at 60° C. for 1 hr. TLC (PE:EA=3:1, Rf=0.6) showed a new spot. The reaction was added H₂O (10 mL) and extracted with EA (10 mL*3). The combined organic layers were washed with sat.NaCl (5 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~40% Ethyl acetate/Petroleum ethergradient @60 mL/min) and concentrated under vacuum to afford Intermediate 1-7 (900 mg, 1.65 mmol, 48.62% yield, 77.143% purity) as yellow solid.

Mass Data: LCMS: Retention time: 0.604 min, [M+H]⁺=420.9, 5-95AB_1 min_220&254.Icm

### Step 6: Synthesis of Intermediate 1-8

To a solution of Intermediate 1-7 (450 mg, 1.07 mmol, 1 eq) was added PMBNH₂ (733.63 mg, 5.35 mmol, 694.07 μL, 5 eq). The mixture was stirred at 80° C. for 6 hr. TLC (PE:EA=3:1, Rf=0.3) showed a new spot. LCMS showed desired MW. The reaction mixture was added DCM (5 mL) and MeOH (2 mL) and purified directly and no work up. The residue was purified by flash silica gel chromatography (ISCO®; 40 g SepaFlash® Silica Flash Column, Eluent of 0~60% Ethylacetate/Petroleum ethergradient @40 mL/min), concentrated under reduced pressure to afford Intermediate 1-8 (200 mg, 383.55 μmol, 35.86% yield) as a yellow solid.

Mass Data: LCMS: Retention time: 0.586 min, [M+H]⁺=522.1, 5-95AB_1 min_220&254.Icm

### Step 7: Synthesis of Intermediate 1-9

To a solution of Intermediate 1-8 (200 mg, 383.55 μmol, 1 eq) in DCM (8 mL) was added TFA (2.20 g, 19.31 mmol, 1.43 mL, 50.35 eq). The mixture was stirred at 40° C. for 12 hr. LCMS showed desired MW. TLC (EA, Rf=0.6) showed a new spot. The pH of the reaction mixture was adjusted to 7-8 with sat.NaHCO₃ and extracted with DCM (2 mL*3). The combined organic layers were washed with sat.NaCl (1 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~100% Ethylacetate/Petroleum ether gradient @60 mL/min) and concentrated under vacuum to afford Intermediate 1-9 (130 mg, 323.96 μmol, 84.46% yield) as yellow solid.

Mass Data: LCMS: Retention time: 0.503 min, [M+H]⁺=402.0, 5-95AB_1 min_220&254.Icm

### Step 8: Synthesis of Intermediate 1-11

To a solution of Intermediate 1-9 (130 mg, 323.96 μmol, 1 eq) in Py (1.5 mL) was added EDCI (310.51 mg, 1.62 mmol, 5 eq) and acrylic acid (70.04 mg, 971.87 μmol, 66.64 μL, 3 eq). The mixture was stirred at 40° C. for 1 hr. LCMS showed desired MW. TLC (PE:EA=1:1, Rf=0.6) showed a spot. The reaction was added H₂O (15 mL) and extracted with EA (10 mL*3). The combined organic layers were washed with sat.NaCl (5 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~100% Ethylacetate/Petroleum ethergradient @60 mL/min) and concentrated under vacuum to afford Intermediate 1-11 (80 mg, 175.69 μmol, 54.23% yield) as white solid.

Mass Data: LCMS: Retention time: 0.528 min, [M+H]⁺=456.0, 5-95AB_1 min_220&254.Icm

### Step 9: Synthesis of Compound 3

To a solution of Intermediate 1-12 (30 mg, 142.76 μmol, 1 eq) in H2O (2 mL) was added Intermediate 1-11 (65.00 mg, 142.76 μmol, 1 eq) and NaOH (1 M, 285.52 μL, 2 eq) then the mixture was stirred at 25° C. for 1 hr. LCMS showed desired MW. The reaction mixture was purified directly and no work up. The residue was purified by reversed phase HPLC(80 g of SepaFlash® Spherical C18, 20-45 μm, 100 Å; mobile phase:A for H2O+0.1% NH3·H2O/H2O and B for acetonitrile; gradient: B 5-95% in 20 min, flow rate: 80 mL/min; column temperature: RT, wavelength: 220 nm/254 nm), concentrated under reduced pressure and lyophilization to afford the residue. Then the residue was purified by prep-HPLC(column: Waters Atlantis T3 150*30 mm*5 um; mobile phase: [H2O(10 mM NH4HCO3)-ACN]; gradient:32%-62% B over 14.0 min) then concentrated under reduced pressure and lyophilization to afford Compound 3 (6.44 mg, 10.38 μmol, 7.27% yield) as a white solid.

Mass Data: LCMS: Retention time=0.090 min,[M−H]+=620.3, 0-60CD_1.5 min_220&254_NEG.Icm; LCMS: Retention time=2.287 min,[M−H]+=620.5, HSS_T3_50_4.6 mm_3.5 μm_5-95CD_4.5minU_M1_1.2_E_NEG.amx

NMR Data: ¹H NMR (400 MHz, DMSO-d6) δ=10.20 (s, 1H), 8.78 (s, 1H), 8.70-8.65 (m, 2H), 7.98 (br d, J=9.4 Hz, 1H), 7.46-7.37 (m, 2H), 7.23-6.92 (m, 7H), 6.17-6.04 (m, 1H), 2.58-2.52 (m, 4H), 2.31 (s, 3H), 1.43-0.63 (m, 11H)

### Example 4

### Biological Assays

Biological assays may be performed in accordance with any relevant literature protocols, including the following support:
Couto Sire M., et al. Cells; 2020. 9: 1408.(doi.org/10.3390/cells9061408)
Urtreger, A. J., et al. Mol Carcinog, 2005. 42(1): 29-39.

### I. In Vitro Assays

### Cell Lines Utilized—

The cells utilized are selected from the following tissue types (and may include others): Breast, Lung, Pancreas, Colon including: HCC1419, MDA-MB-330, MCF-7, NCI-H2342, A-549, BxPC-3, HPAC, T-47D, NCI-H1048, HCT116, SW480.

### Intracellular Accumulation of Boron:

Cells are incubated with selected doses of each of the PIBS of the present invention. The cells are incubated for (1, 2, 4, 6 and 24h) and measurements are carried out at these times. Additionally, the cells are incubated with BPA at the same dose. The cells are washed with 1×PBS and digested with formic acid. Boron content is measured by inductive argon plasma optical emission spectrometry (ICP-AES).

### In-Vitro Biological Effect of the Boron Compounds:

Cells are seeded in 96-well plates in number of 2000 cells/well and incubated with the different PIBS as well as BPA at concentrations including 10 ppm 10B and then irradiated with a neutron beam with irradiation of 1-5 Gy (±10%). Studies are also carried out in the absence of neutron irradiation. Dose response curves are made measuring different biological effects such as: cell survival (by the colorimetric method with MTT); cell death (using a MIX buffer with fluorochromes: Hoechst 33258, DAF and IP) and cell proliferation (by immunocytochemistry through the proliferation marker Ki67 as previously described in Couto above. In addition, the adhesion, invasive and migratory area capacities are studied (Urtreger). With the data obtained, the relative biological effectiveness factors of the neutron beam (RBE) and of each of the borated compounds (CBE) can be used in dosimetric calculations of equivalent physical dose (Gy-Equivalent).

### II. In Vivo Assays

### Animals:

Nude mice of both sexes, 6 to 8 weeks old and weighing 20. 25 g are used. The animals are implanted in the right back flank subcutaneously with 10⁶ cells from the human tumor cell line to be studied. Cells are injected with disposable syringes with a built-in 25 G hypodermic type needle using a maximum volume of 100 pl/mouse. In each animal model, when the tumors are palpable the animals are divided into groups for biodistribution studies. All procedures are carried out in accordance with the Guide for the Use and Care of Laboratory Animals published by the National Institute of Health of the United States (NIH).

### Biodistribution Study

Animals with tumors between 100 and 150 mm3 will be divided into groups and the PIBS compounds are injected intraperitoneally and at different times (1, 2, 4, 6 and 8h) the animals are sacrificed. Samples are taken from different tissues (tumor, blood, skin, liver, spleen, pancreas, kidney, lung) which are weighed and digested with nitric acid and sulfuric acid in a 1:1 ratio. The boron content will be measured by the ICP AES method or LCMS.

### Effect of Complete BNCT Treatment on the Growth of Subcutaneous Tumors:

Mice are inoculated with subcutaneous flank tumor using one of the cell lines described above. When the tumors have values between 100 and 150 mm³, the animals are distributed as follows: 1) BNCT Group I (PIBS compound plus neutrons) (n=4); 2) BNCT II Group (BPA plus neutrons) (n=4); 3) NCT Group (irradiated with single neutrons without boron (n=4); 4) Control Group (without boron and not irradiated) (n=4). The compounds, doses and irradiation time are selected according to the results obtained in the biodistribution studies. Mice are anesthetized with a combination of sedative plus anesthesia consisting of Diazepam 5 mg/kg (s.c) and 20 min then Ketamine 200 mg/kg (s.c). Post treatments, tumor growth are evaluated twice a week. Finally, the mice are sacrificed and histological studies are performed.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. A compound of formula A: 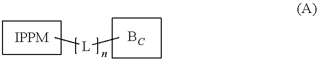
wherein
n is 0 or 1;
IPPM is a molecular structure identified to bind to and modify an intracellular protein pathway at the protein or the DNA encoding the protein associated with the treatment of cancer(s), immunological disorders, and other diseases;
B_{C} is a boron component molecule suitable for use in Boron Neutron Capture Therapy (BNCT); and
L is a linking structure that links IPPM to B_{C}, wherein the identification and selection of the linking position on the IPPM is made to ensure retention of substantial activity of the IPPM to bind and modify the intracellular protein pathway.

2. The compound of claim 1, wherein L is a covalent bond or —S—, —O—, —C(O)—, —C(S)—, —CZ¹₂—, —CZ¹F—, —CF₂—, —NZ¹—, or —S(O)₂—, or a bivalent, saturated or unsaturated, straight or branched C₁₋₂₀ hydrocarbon chain, wherein 0-6 methylene units of L are independently and optionally replaced with —S—, —O—, —C(O)—, —C(S)—, —CZ¹₂—, —CZ¹F—, —CF₂—, —NZ¹—, or —S(O)₂—, or by —C(D)(H)—, —C(D)₂-, —CZ¹F—, —CF₂—, -Cy-, —O—, —N(Z¹)—, —Si(Z¹)₂—, —Si(OH)(Z¹)—, —Si(OH)₂—, —P(O)(OZ¹)—, —P(O)(Z¹)—, —P(O)(NZ¹)—, —S—, —OC(O)—, —C(O)O—, —C(O)—, —S(O)—, —S(O)₂, —N(Z¹)S(O)₂—, —S(O)₂N(Z¹)—, —N(Z¹)C(O)—, —C(O)N(Z¹)—, —OC(O)N(Z¹)—, —N(Z¹)C(O)O—, 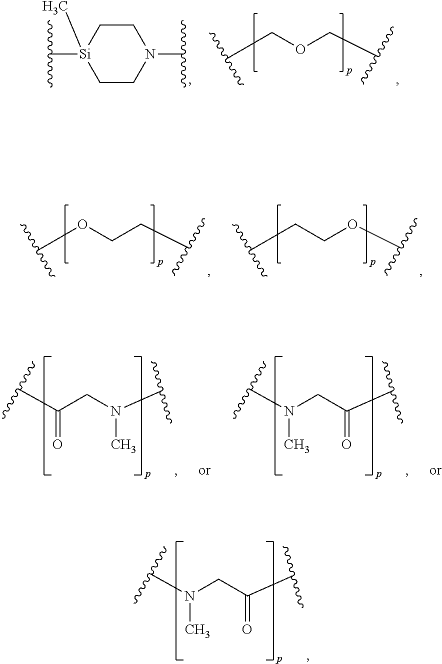
wherein:
each p is independently 0, 1, 2, 3, 4, 5, 6, 7, 8, 9, or 10;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each Z¹ is independently selected from the group consisting of hydrogen, deuterium, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃,
and wherein:
each Z¹ and R is independently selected from hydrogen, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or
two Z¹ or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

3. The compound of claim 1, wherein B_{C} is selected from B₁₂H₁₁,B(OH)₂, Bpin, B(OZ)₂, wherein each Z¹ is as defined above.

4. The compound of claim 1, wherein B_{C} is dodecaborane, which is also referred to as B₁₂H₁₁: 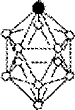
wherein the open circles represents BH and the closed circle represents B.

5. The compound of claim 1, wherein B_{C} is selected from a carborane with mixed amounts of boron and carbon selected from: 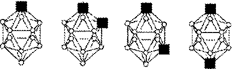
wherein the open circles represent BH and closed square represents carbon (substituted by H or L).

6. The compound of claim 1, wherein B_{C} is selected from a borane selected from the group consisting of hyperclosoborane (BₙHₙ), closoborane ([BₙHₙ]²⁻), nidoborane (BₙHₙ₊₄), arachnoborane (BₙHₙ₊₆), and hyphoborane (BₙHₙ₊₆).

7. The compound of claim 1 having formula (I): 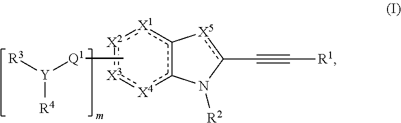
wherein:
each bond denoted by a solid and dashed line is independently a single bond or a double bond;
X¹ is CRS, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
and wherein at least one of X¹, X², X³, and X⁴ is a carbon atom connected to Q;
Q¹ is C═O, C═S, C═CR¹⁴R¹⁵, C═NR⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, or hydrogen, and wherein each R¹ may be further substituted with the substituent -L-B_{c};
each R³ and R⁴ is independently, —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent, and wherein each R³ and R⁴ taken together or independently may be further substituted with the substituent -L-B_{c};
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen;
each R²³ and R²⁴ is independently alkyl, alkenyl, alkynyl, aryl, heteroaryl, or heterocyclyl, each of which is independently substituted or unsubstituted, or hydrogen; and
with the proviso that at least one of R¹, R³, R⁴, or R³ and R⁴ taken together are substituted with the substituent -L-B_{c},
or a pharmaceutically-acceptable salt thereof.

8. The compound of claim 7, comprising the following formula:

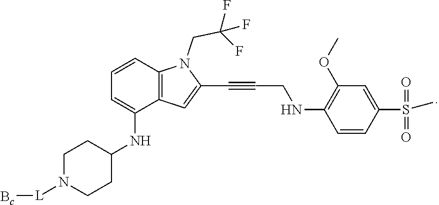

9. The compound of claim 7, comprising the following formula:

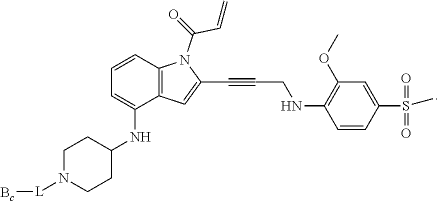

10. The compound of claim 7, comprising the following formula:

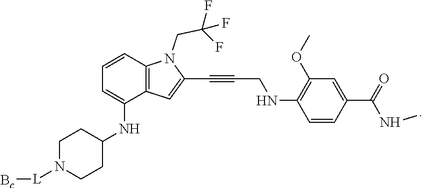

11. The compound of claim 7, comprising the following formula:

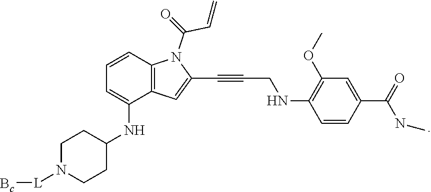

12. The compound of claim 7, comprising the following formula:

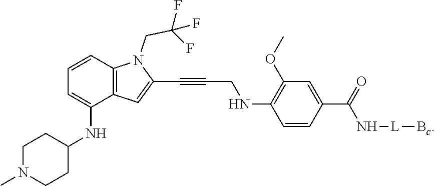

13. The compound of claim 7, comprising the following formula:

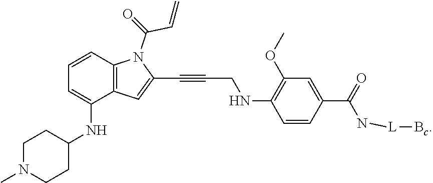

14. The compound of claim 1 having the structure of the formula (II): 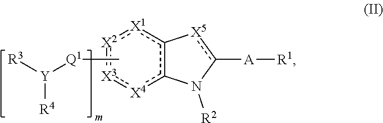
wherein:
each is independently a single bond or a double bond;
X¹ is CRS, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹; —X⁵ is CR¹³, N, or NR¹³;
wherein at least one of X¹, X², X³, and X⁴ is a carbon atom connected to Q¹;
A is a linking group;
Q¹ is C═O, C═S, C═CR¹⁴R¹⁵, C═NR¹⁴, alkylene, alkenylene, or alkynylene, each of which is independently substituted or unsubstituted, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, alkyl, alkenyl, alkynyl, aryl, heteroaryl, or heterocyclyl, each of which is independently substituted or unsubstituted, and wherein each R¹ may be further substituted with the substituent -L-B_{c} or hydrogen;
R³ is H;
R⁴ is heterocyclyl substituted at least with halo-; and may be further substituted with the substituent -L-B_{c};
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is C(O)R²³, —C(O)OR²³, —C(O)NR²³R²⁴, —OR², —SR², —NR²³R²⁴, —NR²C(O)R²⁴, —OC(O)R², alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

15. The compound of claim 1 having the structure of the formula: 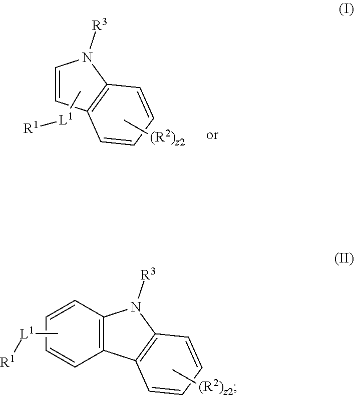
wherein
L¹ is L;
R¹ is B_{c};
R² is independently halogen, —CX²₃, —CHX²₂, —CH₂X², —OCX²₃, —OCH₂X², 7 —OCHX²2, —CN, —SOn2R^{2D}, —SOv2NR^{2A}R^{2B}, —NR^{2C}NR^{2A}R^{2B}, —ONR^{2A}R^{2B},
—NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, 9 —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, 10 —N₃, —NS(O)F₂, —NS(O)FNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, —CCl₃, —CBr₃, —CF₃, —Cl₃, 17 —CHCl₂, —CHBr₂, —CHF₂, —CHI₂, —CH₂Cl, —CH₂Br, —CH₂F, —CH₂I, —CN, —OH, —NH₂, —COOH, 18 —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted
heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is independently an integer from 0 to 4;
m2 and v2 are independently 1 or 2;
z2 is an integer from 0 to 7;
R^{2A} and R^{2B} taken together or independently may be further substituted with the substituent -L-B_{c}; and
R³ is a covalent cysteine modifier moiety.

16. The compound of claim 1 having the structure of the formula: 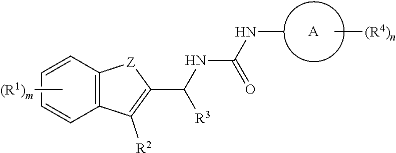
or a pharmaceutically acceptable salt thereof, wherein:
Z is O or NR^{x};
R^{x} is hydrogen, C₁-C₆ alkyl, or C₃-C₆ cycloalkyl;
each R¹ is independently selected from halogen, hydroxyl, cyano, C₁-C₆ alkyl optionally substituted with hydroxyl, and C₃-C₆ cycloalkyl;
m is 0, 1, 2, or 3;
R² is halogen, hydroxyl, C₁-C₆ alkyl optionally substituted with hydroxyl, C₁-C₆ haloalkyl, C₃-C₆ cycloalkyl optionally substituted with 1 or 2 fluoro;
R³ is a C₁-C₆ alkyl, a C₁-C₆ haloalkyl, or a C₃-C₆ cycloalkyl optionally substituted with 1 or 2 substituents independently selected from fluoro and C₁-C₆ alkyl;
Ring A is a 6-10 membered aryl, a C₃-C₈ cycloalkyl, a 5-10 membered heteroaryl, or a 4-10 membered heterocyclyl;
each R⁴ is independently selected from the group consisting of:
(i) halogen,
(ii) C₁-C₆ alkyl optionally substituted with 1 or 2 hydroxyl or —NR^{A}R^{B},
(iii) C₁-C₆ alkoxy optionally substituted with 1-2 substituents independently selected from hydroxyl and C₃-C₆ cycloalkyl,
(iv) C₁-C₆ haloalkyl,
(v) hydroxyl,
(vi) cyano,
(vii) —CO₂H,
(viii) —NR^{A}R^{B},
(ix) ═NR^{A2},
(x) —C(═O)NR^{C}R^{D},
(xi) —SO²(NR^{E}R^{F}),
(xii) —SO₂(C₁-C₆ alkyl),
(xiii) —S(═O)(═NH)(C₁-C₆ alkyl),
(xiv) —C(═O)(C₁-C₆ alkyl),
(xv) —CO₂(C₁-C₆ alkyl),
(xvi) 5-6 membered heteroaryl optionally substituted with C₁-C₆ alkyl,
(xvii) 3-9 membered heterocyclyl optionally substituted with 1 or 2 independently selected R^{G}, and
(xviii) 3-6 membered cycloalkyl optionally substituted with 1 or 2 independently selected RG,
wherein R⁴ is further substituted with the substituent -L-B_{c},
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C₁-C₆ haloalkyl,
(v) —C(═O)(C₁-C₆ alkyl),
(vi) —C(═O)O(C₁-C₆ alkyl),
(vii) —SO₂(C₁-C₆ alkyl),
(viii) 3-6 membered cycloalkyl optionally substituted with hydroxyl, or
(ix) C₁-C₆ alkyl optionally substituted with 1-2 substituents independently selected from hydroxyl, —C(═O)NR^{B2}R^{C2}, 5-6 membered heteroaryl, 3-6 membered cycloalkyl, —SO₂(C₁-C₆ alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{Cl, —SO}₂(C₁-C₆ alkyl), —CO₂H, C₁-C₆ alkyl optionally substituted with hydroxyl, C₁-C₆ alkoxy, and C₁-C₆ haloalkoxy;
each R^{A2}, R^{B2}, and R^{C2} is independently hydrogen or C₁-C₆ alkyl;
each R^{G} is independently selected from the group consisting of: fluoro, cyano, hydroxyl, C₁-C₆ alkyl optionally substituted with hydroxyl, C₁-C₆ alkoxy, —NR^{A1}R^{B1}, ═NR^{A2}, —C(═O)NR^{C1}R^{D1}, —CO²(C₁-C₆ alkyl), C₁-C₆ haloalkyl, C₃-C₆ cycloalkyl, C₁-C₆ haloalkoxy, —SO₂(C₁-C₆ alkyl), and —CO₂H.

17. The compound of claim 16 comprising the following formula:

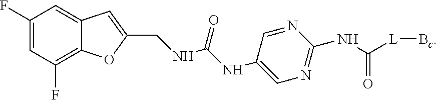

18. The compound of claim 1 having the structure of the formula: 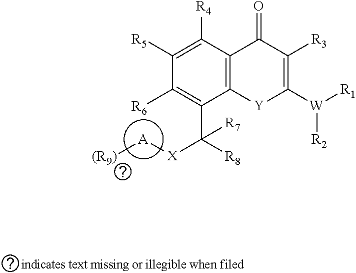
or a prodrug, solvate, enantiomer, stereoisomer, tautomer, or pharmaceutically acceptable salt thereof, wherein:
X is —NR₁₂— or —O—;
Y is —C(R₁)₂—, —O—, —NR₁₁—, or —S—;
WR₁R₂ is a group of the formula: 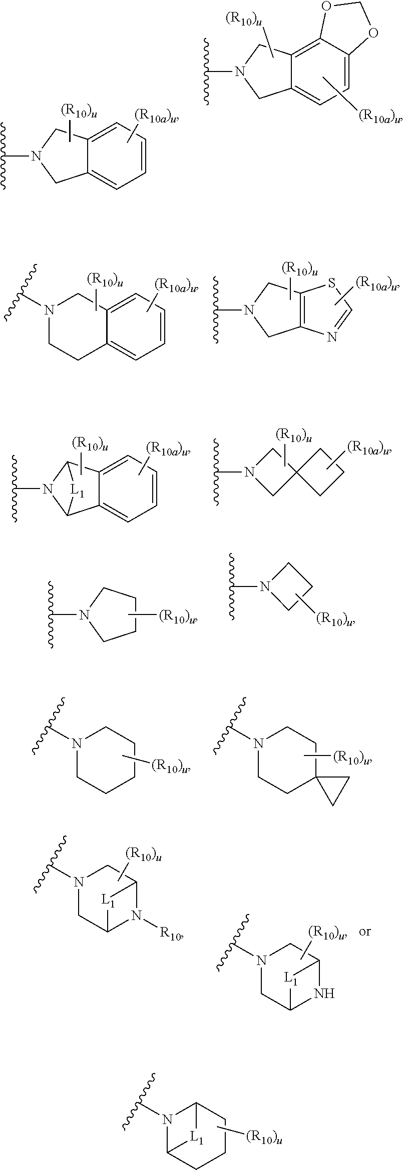
R₁₀ at each occurrence is independently oxo, halogen, —CN, C₁-C₆ alkyl, C₂-C₆ alkenyl, C₂-C₆ alkynyl, C₁-C₆ haloalkyl, C₁-C₆ alkoxy, —(CH₂)ₙ—OR₁₂, —(CH₂)ₙ—N(R₁₂)₂, —(CH₂)ₙ—C(O)R₁₂, —(CH₂)ₙ—C(O)OR₁₂, —(CH₂)ₙ—C(O)N(R₁₂)₂, —(CH₂)ₙ—SO₂R₁₂, —(CH₂)ₙ—O—(CH₂CH₂—O)ᵣR₁₃, C₃-C₁₀ cycloalkyl, heterocycle, —(CH₂)ₙ-aryl, or heteroaryl, wherein the cycloalkyl, heterocycle, aryl, and heteroaryl is optionally substituted with halogen, C₁-C₆ alkyl, C₂-C₆ alkenyl, C₂-C₆ alkynyl, C₁-C₆ haloalkyl, C₁-C₆ alkoxy, C₁-C₆ haloalkoxy, or —(CH₂)ₙ—SO₂R₁₂, which may be further substituted with the substituent -L-B_{c};
R₁₀ₐ at each occurrence is independently halogen, —CN, C₁-C₆ alkyl, C₁-C₆ haloalkyl, C₁-C₆ alkoxy, or —(CH₂)ₙ—OR₁₂;
-L₁- is —(CH₂)— or —(CH₂)₂—;
u at each occurrence is independently 0, 1, 2, 3 or 4;
each R₃, R₄, R₅, and R₆ is independently H, halogen, —CN, C₁-C₆ alkyl, C₂-C₆ alkenyl, C₂-C₆ alkynyl, C₁-C₆ haloalkyl, C₁-C₆ alkoxy, —(CH₂)ₘ—R₁₂, —(CH₂)ₘ—OR₁₂, —(CH₂)ₘ—N(R₁₂)₂, —(CH₂)ₘ—C(O)R₁₂, —(CH₂)ₘ—C(O)OR₁₂, —(CH₂)ₘ—C(O)N(R₁₂)₂, C₃-C₁₀ cycloalkyl, aryl, heterocycle comprising 1-4 heteroatoms selected from O, N, and S, or heteroaryl comprising 1-4 heteroatoms selected from O, N, and S, which may be further substituted with the substituent -L-B_{c};
each R₇ and R₅ is independently H, halogen, —CN, C₁-C₆ alkyl, C₂-C₆ alkenyl, C₂-C₆ alkynyl, C₁-C₆ haloalkyl, or C₁-C₆ alkoxy;
at least one R₉ is —C(O)OR₁₂ and each of the remaining R₉ at each occurrence is independently oxo, ═NR₁₁, halogen, —CN, —NO₂, C₁-C₆ alkyl, C₂-C₆ alkenyl, C₂-C₆ alkynyl, C₁-C₆ haloalkyl, C₁-C₆ alkoxy, —(CH₂)ₘ—N(R₁₂)₂, —(CH₂)ₘ—OR₁₂, —(CH₂)ₘ—CR₁₃(OH)—R₁₂, —(CH₂)ₘ—C(O)R₁₂, —(CH₂)ₘ—C(O)OR₁₂, —(CH₂)ₘ—C(O)N(R₁₂)₂, —(CH₂)ₘ—C(O)N(OH)R₁₂, —(CH₂)ₘ—SO₂R₁₂, —(CH₂)ₘ—SO₂—OR₁₂, —(CH₂)ₘ—SO₂N(R₁₂)₂, —(CH₂)ₘ—P(O)(OR₁₂)₂, —(CH₂)ₘ—P(O)(R₁₂)₂, —(CH₂)ₘ—P(O)(OR₁₃)R₁₂, —(CH₂)ₘ—B(OH)₂, —(CH₂)ₘ—B(R₁₂)₂, —(CH₂)ₘ—O—(CH₂CH₂—O)ᵣR₁₃, —(CH₂)ₘ—NR₁₂—(CH₂CH₂—O)ᵣR₁₃, —(CH₂)ₘ—C(O)—(CH₂CH₂—O)ᵣR₁₃, —(CH₂)ₘ—C(O)O—(CH₂CH₂—O)ᵣR₁₃, —(CH₂)ₘ—C(O)NR₁₂—(CH₂CH₂—O)ᵣR₁₃, —(CH₂)ₘ—C(O)—NR₁₂—SO₂R₁₃, —(CH₂)ₘ—SO₂NR₁₂—C(O)R₁₃, —(CH₂)ₘ—S(O)(NR₁₂)—R₁₃, C₃-C₁₀ cycloalkyl, aryl, heterocycle comprising 1-4 heteroatoms selected from O, N, and S, or heteroaryl comprising 1-4 heteroatoms selected from N, O, and S, wherein the C₁-C₆ alkyl, C₂-C₆ alkenyl, C₂-C₆ alkynyl, C₁-C₆ haloalkyl, C₁-C₆ alkoxy, C₃-C₁₀ cycloalkyl, aryl, heterocycle, or heteroaryl is optionally substituted with one or more oxo, halogen, —CN, —OH, —NH₂, —NO₂, C₁-C₆ alkyl, C₂-C₆ alkenyl, C₂-C₆ alkynyl, C₁-C₆ haloalkyl, or C₁-C₆ alkoxy; or two R₉, together with the atoms to which they are attached form a C₃-C₁₀ cycloalkyl, an aryl, or a heterocycle comprising 1-4 heteroatoms selected from O, N, and S, wherein the cycloalkyl, aryl or heterocycle is optionally substituted with one or more oxo, halogen, —CN, —OH, —NH₂, ═NH, —NO₂, C₁-C₆ alkyl, C₂-C₆ alkenyl, C₂-C₆ alkynyl, C₁-C₆ haloalkyl, or C₁-C₆ alkoxy, which may be further substituted with the substituent -L-B_{c};
R₁ is H, C₁-C₈alkyl, C₂-C₆ alkenyl, or C₂-C₆ alkynyl;
each R₁₂ and R₁₃ at each occurrence is independently H, C₁-C₆ alkyl, C₂-C₆ alkenyl, C₂-C₆ alkynyl, C₁-C₆ haloalkyl, C₁-C₆ alkoxy, —(CH₂)_{q}—O—C(O)—(CH₂)ᵣ R₁₄, —(CH₂)_{q}—NH—C(O)—(CH₂)ᵣR₁₄, —(CH₂)_{q}—O—C(O)—(CH₂)ᵣ—OR₁₄, —(CH₂)_{q}—NH—C(O)—(CH₂)ᵣOR₁₄, —(CH₂)_{q}—O—(CH₂)ᵣR₁₄, —(CH₂)_{q}—NH—(CH₂)ᵣR₁₄, —(CH₂)_{q}—O—(CH₂)ᵣ OR₁₄, —(CH₂)_{q}—NH—(CH₂)ᵣOR₁₄, C₃-C₁₀ cycloalkyl, heterocycle comprising 1-4 heteroatoms selected from O, N, and S, —(CH₂)_{q}-aryl, or heteroaryl comprising 1-4 heteroatoms selected from N, O, and S, wherein the cycloalkyl, heterocycle, aryl, and heteroaryl are optionally substituted with one or more halogen, C₁-C₆ alkyl, C₁-C₆ haloalkyl, C₁-C₆ alkoxy, or C₁-C₆ haloalkoxy;
Ring A is C₃-C_{1O} cycloalkyl, aryl, heterocycle comprising 1-4 heteroatoms selected from N, O, and S, or heteroaryl comprising 1-4 heteroatoms selected from N, O, and S; 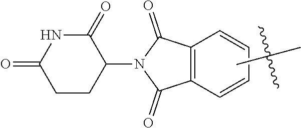
each n, m, q, or r, is independently at each occurrence 0, 1, 2, 3, 4, 5, or 6; and s is 1, 2, 3, 4, 5, or 6.
with the proviso that at least one of R₃, R₄, R₅, R₆, R₉, or R₁₀ are substituted with the substituent -L-B_{c}.

19. The compound of claim 18, comprising the following formula:

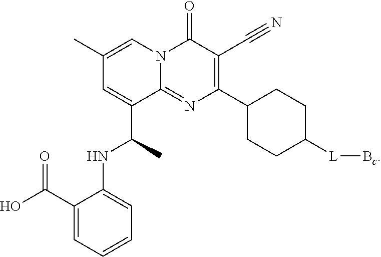

20. The compound of claim 1 having the structure of the formula: 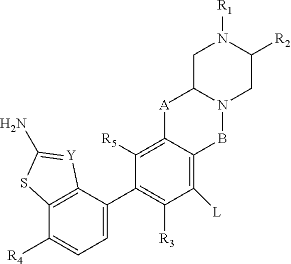
wherein:
A is —OCH₂—, —N(R₆)CH₂, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, H, or a group of the formula 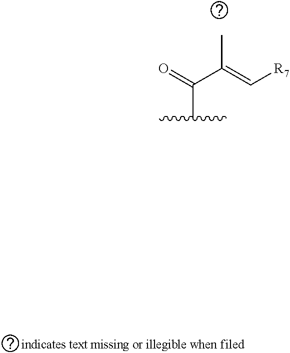
R₂ is H, methyl, or —CH₂CN;
R₃ and R5 are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
L is a linking structure described herein throughout and substituted with B_{c} forming the substituent -L-B_{c};
R₇ is H, halogen, —NR₁₁R₁₂, —CH₂NR₁₁R₁₂, —Cₗ₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C1-4 alkyl, or —O—C1-4 alkyl;
R₁₁ and R₁₂ are each independently H, —C₁₋₄ alkyl, or —C₁₋₄ heteroalkyl, wherein R₁₁ and R₁₂ may combine to form a cycloheteroalkyl; and
R₁₃ is independently at each occurrence —N—C₁₋₄ alkyl, H,
or a pharmaceutically acceptable salt thereof.

21. The compound of claim 20 comprising the following formula:

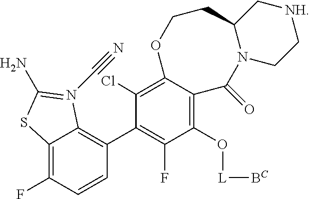

22. The compound of claim 20 comprising the following formula:

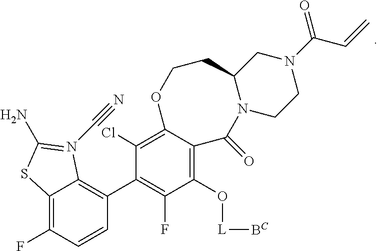

23. The compound of claim 1 having the structure of the formula: 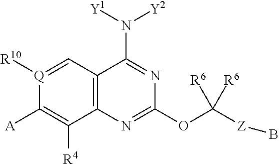
or a pharmaceutically acceptable salt thereof,
wherein:
A is aryl or heteroaryl, wherein the aryl or the heteroaryl is optionally substituted with 1-4 R¹;
B is: 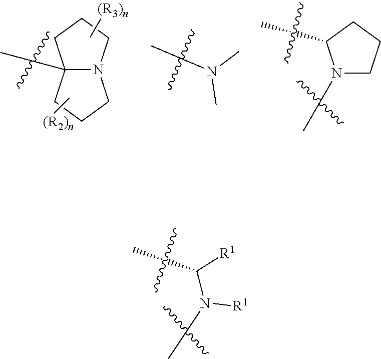
substituted with the substituent -L-B_{c};
Y¹ is hydrogen, hydroxy, halogen, Z—SO₂—NH₂, Z—OH, C₁-C₄ alkyl, Z—C3-C6 cycloalkyl optionally substituted with 1-4 R⁹, Z-heteroaryl optionally substituted with 1-4 R⁸, Z-aryl optionally substituted with 1-4 R⁸, Z—C(O)—NH₂, and Z-heterocycle optionally substituted with 1-2 oxo (═O) or oxo-containing substituent, and optionally further substituted with 1-2 R⁸,
Z is a bond, —C1-C4 alkyl-, —NH—, —N(C1-C3 alkyl)- or cyclopropyl-CH₂—;
Y² is hydrogen or C1-C4 alkyl;
or Y¹ and Y²join to form a monocyclic or bicyclic substituted or unsubstituted heterocyclyl, or:
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —OC(O)N(R⁵)₂, —CO₂R⁵, or —CO₂N(R⁵)₂;
each R³ is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —COC(O)N(R⁵)₂, —CO₂R⁵, —CO₂N(R⁵)₂, or R³ is 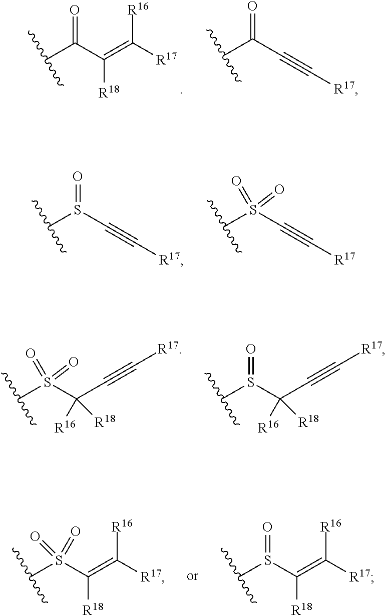
and
wherein R¹⁶, R¹⁷, and R¹⁸ are independently hydrogen, oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen or C1-C3 alkyl;
each R⁵ is independently hydrogen or C1-C3 alkyl;
each R⁸ is independently hydrogen, hydroxy, C1-C4 hydroxyalkyl or heteroaryl;
each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, aryl, —CH₂—S(O)₂NH₂, or heteroaryl optionally independently substituted with 1-2 C1-C3 alkyl, —CN or C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;
each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;
Q is N, C or O, wherein if Q is C the 6-membered ring that includes Q is aromatic, and wherein if Q is O the 6-membered ring that includes Q is an oxane;
each n is 0-3;
is 1-6; and
p is 1-8.

24. The compound of claim 23 comprising the following formula: 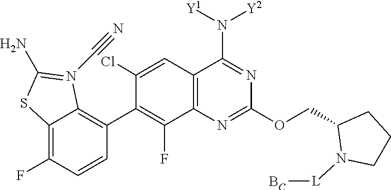
wherein Y¹ and Y² join to form a monocyclic or bicyclic substituted or unsubstituted heterocyclyl.

25. The compound of claim 23 comprising the following formula:

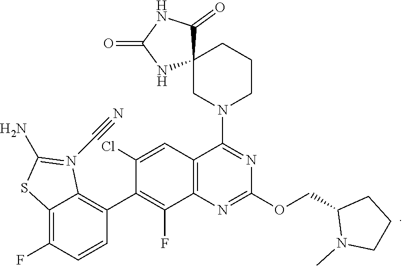

26. The compound of claim 23 comprising the following formula:

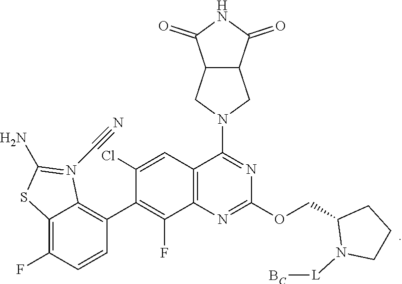

27. The compound of claim 1 wherein -L-B_{C} is selected from the group consisting of:

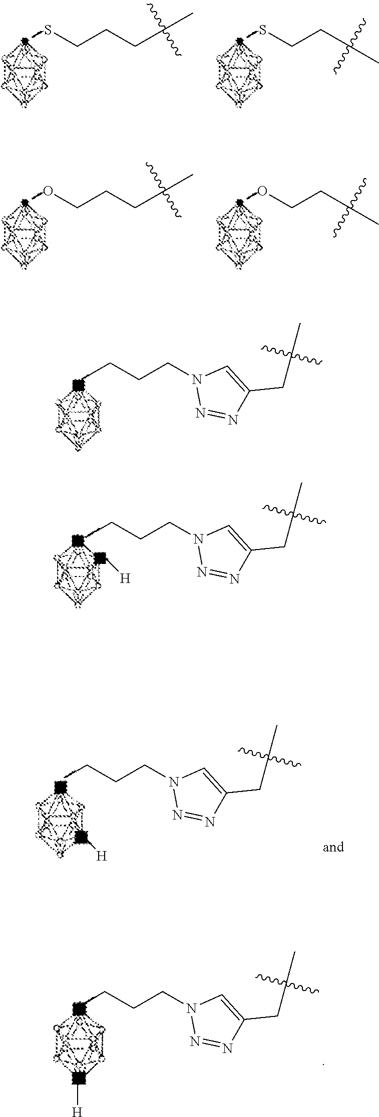

28. A pharmaceutical composition comprising a compound of claim 1, or a pharmaceutically acceptable derivative thereof, and a pharmaceutically acceptable carrier, adjuvant, or vehicle.

29. A method of treating an intracellular protein mediated disorder, disease, or condition in a patient comprising administering to said patient a compound of claim 1.

30. The method of claim 29 further comprising the step of irradiating the patient with neutrons.

31. The method of claim 29, wherein the intracellular protein mediated disorder is associated with the signaling pathway selected from the group consisting of the RAS/MAPK pathway, the PI3K/AKT/mTOR pathway, the Notch pathway, the Wnt/β-catenin pathway, the P53 pathway, the TGF-β pathway, and the Hippo YAP/TAZ pathway.

32. The method of claim 29, wherein the intracellular protein mediated disorder is a cancer.

33. The method of claim 32, wherein the cancer is selected from the group consisting of a solid cancer, hematological cancer, lung cancer, pancreatic cancer, colorectal cancer, ovarian cancer, a cancer which is a metastatic cancer, or a cancer which is relapsed, a cancer which is refractory, a squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

34. The method of claim 32, wherein the cancer is selected from the group consisting of acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.
